(19) United States
(12) Patent Application Publication
(10) Pub. No.: US 2003/0191110 A1
(43) Pub. Date: Oct. 9, 2003

(54) Modulators of the cholesterol biosynthetic pathway

(76) Inventors: Martyn Botfield, Boston, MA (US); Fiona McDonald, Berlin (DE); Joern Kraetzschmar, Berlin (DE); Bernhard Lindenthal, Berlin (DE); Bertold Kreft, Berlin (DE)

Correspondence Address: VERTEX PHARMACEUTICALS INC., 130 WAVERLY STREET, CAMBRIDGE, MA, 02139-4242, US

(21) Appl. No.: 10/286,416

(22) Filed: Nov. 1, 2002

Related U.S. Application Data
(60) Provisional application No. 60/344,485, filed on Nov. 1, 2001.

(51) Int. Cl. A61K 31/553; A61K 31/554; A61K 31/551; A61K 31/55; A61K 31/541; A61K 31/5377; A61K 31/496; A61K 31/519; A61K 31/52

(52) U.S. Cl. 514/211.15; 514/217.04; 514/217.05; 514/217.06; 514/217.07; 514/217.08; 514/218; 514/228.2; 514/228.5; 514/234.2; 514/234.5; 514/241; 514/252.16; 514/248; 514/249; 514/252.02; 514/255.03; 514/255.05; 514/253.04; 514/252.17; 514/263.22; 514/263.2; 514/266.2; 514/266.22; 514/314; 514/307; 514/300; 514/340; 514/341; 514/342; 514/365; 514/397; 514/381; 514/374

(57) ABSTRACT

The present invention relates to methods for modulating the cholesterol biosynthetic pathway. The level of cholesterol in the body is linked to numerous pathological states. The methods of the present invention alter the transcription levels of genes involved in the cholesterol biosynthesis. The methods of the present invention can used for treating diseases mediated by the cholesterol biosynthetic pathway.

### CROSS-REFERENCE TO RELATED APPLICATIONS

The present application claims the benefit of U.S. Provisional Application No. 60/344,485, filed Nov. 1, 2001.

### TECHNICAL FIELD OF THE INVENTION

The present invention relates to methods for modulating the cholesterol biosynthetic pathway. The level of cholesterol in the body is linked to numerous pathological states. The methods of the present invention alter the transcription levels of genes involved in the cholesterol biosynthesis. The methods of the present invention can be used for treating diseases mediated by the cholesterol biosynthetic pathway.

### BACKGROUND OF THE INVENTION

The largest pool and highest concentration of cholesterol in the body exists in the brain (Maekawa et al., 1999, J. Biol. Chem. 274, 21369-21374). Cholesterol homeostasis in the brain is unique in that neurons are entirely dependent on de novo biosynthesis and cannot take up cholesterol from the bloodstream. Disruption of cholesterol homeostasis plays a major role in the pathogenesis of diseases including Creutzfeld-Jakob and other prion diseases (Taraboulos et al, 1995, J. Cell Biol. 129, 121-132), Niemann-Pick Type C disease (Henderson et al, 2000, J. Biol. Chem. 275, 20179-20187), Smith-Lemli-Opitz syndrome (Tint et al, 1994, N. Engl. J. Med. 330, 107-113), Tangier disease, and possibly in AIDS-induced peripheral neuropathy and dementia (Falkenbach et al, 1990, Med. Hypotheses 33, 57-61).

In scrapie-infected cultured cells, depletion of cellular cholesterol by the HMGCOA reductase inhibitor lovastatin slowed the normal degradation of the PrPC isoform and its conversion to the pathogenic PrPSc isoform. This effect was shown to be cholesterol-related and not due to depletion of other cellular components for which mevalonate is a biosynthetic precursor. The rate of synthesis of PrPC was not reduced by lovastatin. A chimeric form of PrPC that cannot associate with cholesterol-rich membrane domains, but is directed instead to clathrin-coated pits, was not converted to PrPSc. It is possible that association of PrPC with cholesterol-rich lipid “rafts” increases the local concentration and makes it easier for the PrPSc form to associate with it and start a “chain reaction” of conversion to the pathogenic form. But this has not been proven thus far (Taraboulos, A. et al., (1995) J. Cell Biol., 129(1), 121-32).

Much of the toxicity caused by neurotoxic insults results from leakage of excitatory neurotransmitters out of the damaged neurons. Cholesterol decreases the fluidity of cell membranes. An increase in cholesterol biosynthesis, a decrease in cholesterol metabolism, or improved ability to re-uptake “scavenged” cholesterol released by neuronal damage could limit this leakage. Cholesterol is crucial for modulating cell membrane fluidity. This controls the degree of “leakiness” of cells and affects the release of toxic excitatory neurotransmitters upon injury.

A pathogenic form of the prion protein, as well as a pathogenic Alzheimer's disease peptide, caused cation-selective channels to form in cultured neuronal cells. This increased the influx of calcium ions, which provides a reasonable mechanism for the toxicity of these peptides in both AD and in prion diseases. (Loss of control of cellular calcium levels leads to cell death.) Cholesterol protected against this toxicity in the case of the AD peptide; protection by cholesterol was not tested for the prion protein, but they have structural similarities that suggest that increased cellular cholesterol would be protective for prion diseases as well. Both the AD and prion peptides can only form channels in acidic phospholipid bilayers and not in cholesterol-containing segments of the membrane. This suggests that increasing the cholesterol content of neuronal membranes might be protective against prion diseases. Kawahara, M., Kuroda et al., J Biol Chem 275(19), 14077-83. Well known prion diseases include Creutzfeldt-Jakob disease of man displaying sporadic, inherited and infectious forms, bovine spongiform encephalopathy and scrapie of sheep. Haltia, M., Ann. Med. 2000, 32, pp. 493-500.

The oxytocin receptor requires a specific interaction with cholesterol in order to function (Gimpl et al., 1997, Biochemistry 36, 10959-10974). Oxytocin and related neuropeptides are believed to play a role in learning (Moore et al., 1991, Neurosurg. Rev. 14, 97-110). Mutations in Δ⁷-sterol reductase, an enzyme of cholesterol biosynthesis, have been linked to Smith-Lemli-Opitz syndrome, a fatal disorder in which brain development is deranged (Fitzky et al., 1998, Proc. Natl. Acad. Sci. U.S.A. 95, 8181-8186). Cholesterol is also essential for the assembly of myelin (Simons et al, 2000, J. Cell Biol. 151, 143-153).

Thus, the level of cholesterol in the body is linked to numerous pathological states. Consequently, there is a need for the discovery and design of methods for modulating the cholesterol biosynthesis in the body. Such a modulation will enable the control of cholesterol levels, thus providing a method of treating diseases mediated by cholesterol biosynthesis.

### SUMMARY OF THE INVENTION

The present invention provides a method of modulating cholesterol biosynthesis in a mammal by administering to said mammal a composition comprising:

(i) a pharmaceutically effective compound of a formula (I):

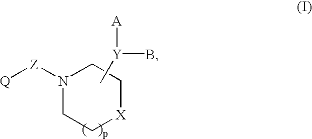

wherein:

each Q is a monocyclic, bicyclic or tricyclic ring system wherein in said ring system:

a. each ring is independently partially unsaturated or fully saturated;

b. each ring comprises 3 to 7 ring atoms independently selected from C, N, O or S;

c. no more than 4 ring atoms in Q are selected from N, O or S;

d. any S is optionally replaced with S(O) or S(O)₂;

e. at least one ring comprises a N ring atom that is substituted with R¹;

f. one to five hydrogen atoms in Q are optionally and independently replaced with halo, —OH, ═O, ═N—OR¹, (C₁-C₆)-straight or branched alkyl, Ar-substituted-(C₁-C₆)-straight or branched alkyl, (C₂-C₆)-straight or branched alkenyl or alkynyl, Ar-substituted-(C₂-C₆)-straight or branched alkenyl or alkynyl, O—(C₁-C₆)-straight or branched alkyl, O-[(C₁-C₆)-straight or branched alkyl]-Ar, O—(C₂-C₆)-straight or branched alkenyl or alkynyl, O-[(C₂-C₆)-straight or branched alkenyl or alkynyl]-Ar, or O—Ar; and

g. Q is not an indole or a pyroglutamic moiety;

wherein

each R¹ is independently selected from (C₁-C₁₀)-straight or branched alkyl, Ar-substituted-(C₁-C₁₀)-straight or branched alkyl, (C₂-C₁₀)-straight or branched alkenyl or alkynyl, or Ar-substituted-(C₂-C₁₀)-straight or branched alkenyl or alkynyl; wherein

one to two CH₂ groups of said alkyl, alkenyl, or alkynyl chains in R¹ are optionally and independently replaced with O, S, S(O), S(O)₂, C(O) or N(R²), wherein when R¹ is bound to nitrogen, the CH₂ group of R¹ bound directly to said nitrogen cannot be replaced with C(O);

Ar is selected from phenyl, 1-naphthyl, 2-naphthyl, indenyl, azulenyl, 2-furyl, 3-furyl, 2-thienyl, 3-thienyl, 2-pyridyl, 3-pyridyl, 4-pyridyl, pyrrolyl, oxazolyl, thiazolyl, imidazolyl, pyraxolyl, pyrazolinyl, pyraolidinyl, isoxazolyl, isothiazolyl, 1,2,3-oxadiazolyl, 1,2,3-triazolyl, 1,3,4-thiadiazolyl, 1,2,4-triazolyl, 1,2,4-oxadiazolyl, 1,2,4-thiadiazolyl, 1,2,3-thiadiazolyl, benoxazolyl, pyridazinyl, 2-pyrimidinyl, 4-pyrimidinyl, 5-pyrimidinyl, pyrazinyl, 1,3,5-triazinyl, 1,3,5-trithianyl, indolizinyl, indolyl, isoindolyl, 3H-indolyl, indolinyl, benzo[b]furanyl, benzo[b]thiophenyl, 1H-indazolyl, benzimidazolyl, benzthiazolyl, purinyl, 4H-quinolizinyl, quinolinyl, 1,2,3,4-tetrahydroisoquinolinyl, isoquinolinyl, 1,2,3,4-tetrahydroquinolinyl, cinnolinyl, phthalazinyl, quinazolinyl, quinoxalinyl, 1,8-naphthyridinyl, or any other chemically feasible monocyclic or bicyclic ring system, wherein each ring consists of 5 to 7 ring atoms and wherein each ring comprises 0 to 3 heteroatoms independently selected from N, O, or S, wherein

each Ar is optionally and independently substituted with one to three substituents selected from halo, hydroxy, nitro, ═O, —SO₃H, trifluoromethyl, trifluoromethoxy, (C₁-C₆)-straight or branched alkyl, (C₁-C₆)-straight or branched alkenyl, O-[(C₁-C₆)-straight or branched alkyl], O-[(C₁-C₆)-straight or branched alkenyl], O-benzyl, O-phenyl, 1,2-methylenedioxy, —N(R³) (R⁴), carboxyl, N-(C₁-C₆-straight or branched alkyl or C₂-C₆-straight or branched alkenyl) carboxamides, N,N-di-(C₁-C₆-straight or branched alkyl or C₂-C₆-straight or branched alkenyl) carboxamides, N-(C₁-C₆-straight or branched alkyl or C₂-C₆-straight or branched alkenyl) sulfonamides, or N,N-di-(C₁-C₆-straight or branched alkyl or C2-C6-straight or branched alkenyl) sulfonamides;

each of R³ and R⁴ are independently selected from (C₁-C₆)-straight or branched alkyl, (C₂-C₆)-straight or branched alkenyl or alkynyl, hydrogen, phenyl or benzyl; or wherein R³ and R⁴ are taken together with the nitrogen atom to which they are bound to form a 5-7 membered heterocyclic ring;

each R² is independently selected from hydrogen, (C₁-C₆)-straight or branched alkyl, or (C₂-C₆)-straight or branched alkenyl or alkynyl;

X is selected from C(R²)₂, N(R²), N, O, S, S(O), or S(O)₂

Y is selected from a bond, —O—, (C₁-C₆)-straight or branched) alkyl, or (C₂-C₆)-straight or branched) alkenyl or alkynyl; wherein Y is bonded to the depicted ring via a single bond or a double bond; and wherein one to two of the CH₂ groups of said alkyl, alkenyl, or alkynyl is optionally and independently replaced with O, S, S(O), S(O)₂, C(O) or N(R²);

Z is —C(O)— or —CH₂—

p is 0, 1 or 2;

each of A and B is independently selected from hydrogen or Ar; or one of A or B is absent; and

wherein two carbon ring atoms in the depicted ring structure are optionally linked to one another via a C₁-C₄ straight alkyl or a C₂-C₄ straight alkenyl to create a bicyclic moiety; and

(ii) a pharmaceutically acceptable carrier.

The present invention also provides methods of treating a disease mediated by cholesterol biosynthesis.

The present invention also provides a method of treating Creutzfeld-Jakob disease, Kuru, Gerstmann-Straussler-Scheinker disease and fatal familial insomnia. The present invention is also useful in treating veterinary diseases such as BSE, Scrapie and transmissible mink encephalopathy.

### DETAILED DESCRIPTION OF THE INVENTION

The present invention provides a method of modulating cholesterol biosynthesis in a mammal by administering to said mammal a composition comprising:

(i) a pharmaceutically effective compound of a formula (I):

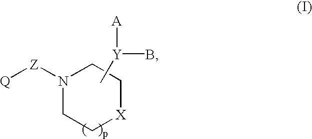

wherein:

each Q is a monocyclic, bicyclic or tricyclic ring system wherein in said ring system:

a. each ring is independently partially unsaturated or fully saturated;

b. each ring comprises 3 to 7 ring atoms independently selected from C, N, O or S;

c. no more than 4 ring atoms in Q are selected from N, O or S;

d. any S is optionally replaced with S(O) or S(O)₂;

e. at least one ring comprises a N ring atom that is substituted with R¹;

f. one to five hydrogen atoms in Q are optionally and independently replaced with halo, —OH, ═O, ═N—OR¹, (C₁-C₆)-straight or branched alkyl, Ar-substituted-(C₁-C₆)-straight or branched alkyl, (C₂-C₆)-straight or branched alkenyl or alkynyl, Ar-substituted-(C₂-C₆)-straight or branched alkenyl or alkynyl, O—(C₁-C₆)-straight or branched alkyl, O-[(C₁-C₆)-straight or branched alkyl]-Ar, O—(C₂-C₆)-straight or branched alkenyl or alkynyl, O-[(C₂-C₆)-straight or branched alkenyl or alkynyl]-Ar, or O—Ar; and

g. Q is not an indole or a pyroglutamic moiety, wherein

each R¹ is independently selected from (C₁-C₁₀)-straight or branched alkyl, Ar-substituted-(C₁-C₁₀)-straight or branched alkyl, cycloalkyl-substituted-(C₁-C₁₀)-straight or branched alkyl, (C₂-C₁₀)-straight or branched alkenyl or alkynyl, or Ar-substituted-(C₂-C₁₀)-straight or branched alkenyl or alkynyl; wherein

one to two CH₂ groups of said alkyl, alkenyl, or alkynyl chains in R¹ are optionally and independently replaced with O, S, S(O), S(O) 2, C(O) or N(R²), wherein when R¹ is bound to nitrogen, the CH₂ group of R¹ directly bound to said nitrogen cannot be replaced with C(O);

Ar is selected from phenyl, 1-naphthyl, 2-naphthyl, indenyl, azulenyl, 2-furyl, 3-furyl, 2-thienyl, 3-thienyl, 2-pyridyl, 3-pyridyl, 4-pyridyl, pyrrolyl, oxazolyl, thiazolyl, imidazolyl, pyraxolyl, pyrazolinyl pyraolidinyl, isoxazolyl, isothiazolyl, 1,2,3-oxadiazolyl, 1,2,3-triazolyl, 1,3,4-thiadiazolyl, 1,2,4-triazolyl, 1,2,4-oxadiazolyl, 1,2,4-thiadiazolyl, 1,2,3-thiadiazolyl, benoxazolyl, pyridazinyl, 2-pyrimidinyl, 4-pyrimidinyl, 5-pyrimidinyl, pyrazinyl, 1,3,5-triazinyl, 1,3,5-trithianyl, indolizinyl, indolyl, isoindolyl, 3H-indolyl, indolinyl, benzo[b]furanyl, benzo[b]thiophenyl, 1H-indazolyl, benzimidazolyl, benzthiazolyl, purinyl, 4H-quinolizinyl, quinolinyl, 1,2,3,4-tetrahydroisoquinolinyl, isoquinolinyl, 1,2,3,4-tetrahydroquinolinyl, cinnolinyl, phthalazinyl, quinazolinyl, quinoxalinyl, 1,8-naphthyridinyl, or any other chemically feasible monocyclic or bicyclic ring system, wherein each ring consists of 5 to 7 ring atoms and wherein each ring comprises 0 to 3 heteroatoms independently selected from N, O, or S, wherein

each Ar is optionally and independently substituted with one to three substituents selected from halo, hydroxy, nitro, ═O, —SO₃H, trifluoromethyl, trifluoromethoxy, (C₁-C₆)-straight or branched alkyl, (C₁-C₆)-straight or branched alkenyl, O-[(C₁-C₆)-straight or branched alkyl], O-[(C₁-C₆)-straight or branched alkenyl], O-benzyl, O-phenyl, 1,2-methylenedioxy, —N(R³)(R⁴), carboxyl, N-(C₁-C₆-straight or branched alkyl or C₂-C₆-straight or branched alkenyl) carboxamides, N,N-di-(C₁-C₆-straight or branched alkyl or C₂-C₆-straight or branched alkenyl) carboxamides, N-(C₁-C₆-straight or branched alkyl or C₂-C₆-straight or branched alkenyl) or sulfonamides, N,N-di-(C₁-C₆-straight or branched alkyl or C₂-C₆-straight or branched alkenyl) sulfonamides;

each of R³ and R⁴ are independently selected from (C₁-C₆)-straight or branched alkyl, (C₂-C₆)-straight or branched alkenyl or alkynyl, hydrogen, phenyl or benzyl; or wherein R³ and R⁴ are taken together with the nitrogen atom to which they are bound to form a 5-7 membered heterocyclic ring;

each R² is independently selected from hydrogen, (C₁-C₆)-straight or branched alkyl, or (C₂-C₆)-straight or branched alkenyl or alkynyl;

X is selected from C(R²)₂, N(R²), N, O, S, S(O), or S(O)₂

Y is selected from a bond, —O—, (C₁-C₆)-straight or branched) alkyl, or (C₂-C₆)-straight or branched) alkenyl or alkynyl; wherein Y is bonded to the depicted ring via a single bond or a double bond; and wherein one to two of the CH₂ groups of said alkyl, alkenyl, or alkynyl is optionally and independently replaced with O, S, S(O), S(O)₂, C(O) or N(R);

p is 0, 1 or 2;

Z is —C(O)— or —CH₂—;

each of A and B is independently selected from hydrogen or Ar; or one of A and B is absent; and

wherein two carbon ring atoms in the depicted ring structure may be linked to one another via a C₁-C₄ straight alkyl or a C₂-C₄ straight alkenyl to create a bicyclic moiety; and

(ii) a pharmaceutically acceptable carrier.

The term “ring atom”, as used herein, refers to a backbone atom that makes up the ring. Such ring atoms are selected from C, N, O or S and are bound to 2 or 3 other such ring atoms (3 in the case of certain ring atoms in a bicyclic ring system). The term “ring atom” does not include hydrogen.

It will be readily apparent to those of skill in the are that the terms “alkyl” and “alkenyl” when used in the definition of Y represent those portions of an aliphatic moiety for which proper valence is completed by the moities bound to Y (i.e., at one end, the ring atom to which Y is bound; and at the other end, A and B). Thus, as an example, for the purposes of this invention, Y is considered a C₂ alkyl in each of the following structures (the moiety representing Y being shown in bold):

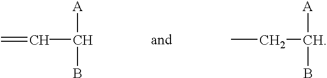

According to a preferred embodiment of the present invention, Q in a compound of formula (I) is selected from a 5 to 6 membered partially unsaturated or fully saturated heterocyclic ring containing a single nitrogen ring atom and four to five carbon ring atoms, wherein said ring is optionally fused to a three-membered ring. Even more preferred is when Q is piperidyl, pyrrolidyl or

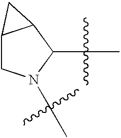

(3-Azabicyclo[3.1.0]hexyl). Most preferred is when Q is piperidyl or pyrrolidyl optionally substituted at one of the ring carbons with phenyl, methyl or hydroxy or Q is 3-Azabicyclo[3.1.0]hexyl.

According to another preferred embodiment, R¹ is selected from (C₁-C₆)-straight alkyl, (C₁-C₆)-straight alkyl-Ar, (C₁-C₆)-straight alkyl-cycloalkyl, (C₃-C₆)-straight or branched alkenyl, or (C₃-C₆)-straight or branched alkenyl-Ar. Even more preferred is when R¹ is selected from methyl, ethyl, —CH₂-phenyl, —CH₂-methylphenyl, —CH₂-methoxyphenyl, —CH₂-fluorophenyl, —CH₂-difluorophenyl, —CH₂—CH₂-phenyl, —CH₂-cyclopropyl, —CH₂—CH═C(CH₃)₂, —CH₂—CH═CH₂, or —CH₂—CH═CH-phenyl.

In yet another preferred embodiment, p is 0 or 1; and X is C or N.

In another preferred embodiment of the compound of formula (I), Y is a bond, —O—, —CH<, or ═CH<.

According to another preferred embodiment, one of A or B is absent or selected from hydrogen, phenyl, chlorophenyl, dichlorophenyl, fluorophenyl, or difluorophenyl and the other of A or B is selected from phenyl, chlorophenyl, dichlorophenyl, fluorophenyl, or difluorophenyl.

Some of the more preferred embodiments of this invention are the compounds listed in Table 1 and Table 2, below and the compounds set forth in the Examples.

TABLE 1

| | |
|---|---|
| 1 | 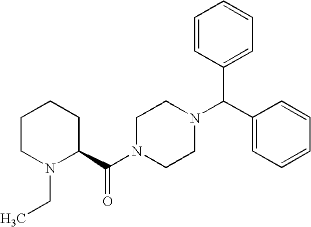 |
| 2 | 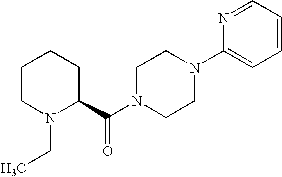 |
| 4 | 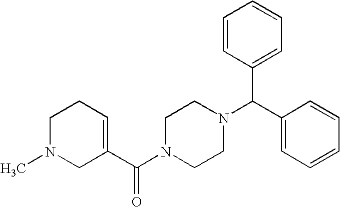 |
| 7 | 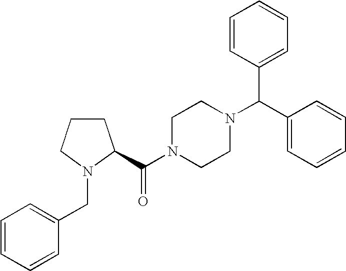 |
| 8 | 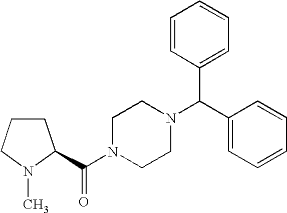 |
| 11 | 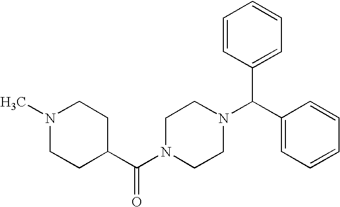 |
| 15 | 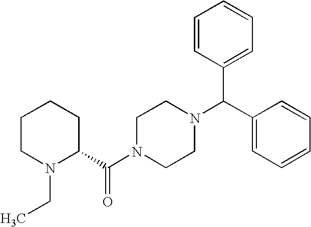 |
| 16 | 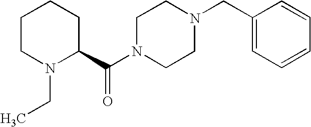 |

TABLE 2

| | |
|---|---|
| 17 | 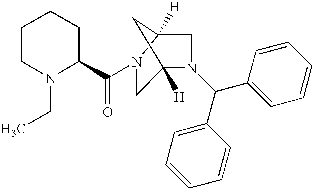 |
| 20 | 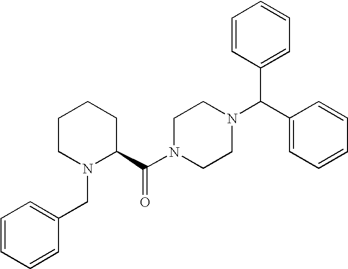 |
| 21 | 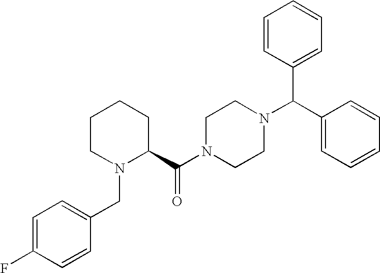 |
| 24 | 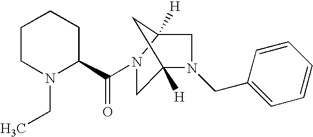 |
| 25 | 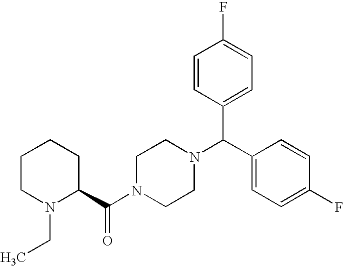 |
| 26 | 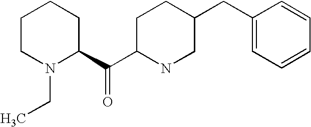 |
| 27 | 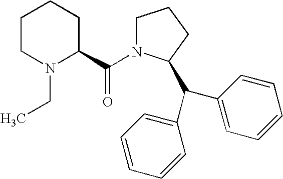 |
| 28 | 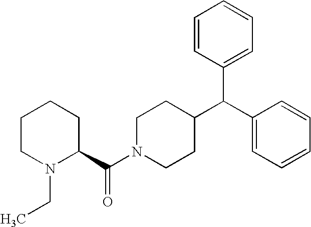 |
| 29 | 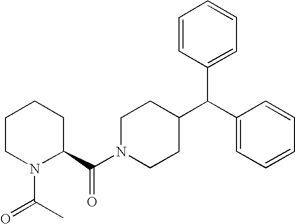 |

Even more preferred are compounds 1, 7, 15, 20, 21, 26, 28, 29, 30, 39, 41, 42, 44, 47, 48, 49, 52, 58, 60, 65, 69, 84, 85, 86, 90, 100, 101, 102, 103, 205, 206, 221, 223, 225, 238, 240, 242, 246, 255, 260, 261, 262, 263, 265, 267, 268, 271, 273, 275, 276, 277, 278, or 279.

The compounds of formula (I) may be stereoisomers, geometric isomers or stable tautomers. The invention envisions all possible isomers, such as E and Z isomers, S and R enantiomers, diastereoisomers, racemates, and mixtures of those.

Without wishing to be bound by theory, applicants believe that the methods of the present invention operate by, inter alia, altering the transcription levels of genes responsible for the biosynthesis of cholesterol. Such an alteration affects the levels of cholesterol and, consequently, cholesterol metabolites in the mammal.

The compounds of the present invention may be readily prepared using known synthetic methods. For example, compounds of formula (I) may be prepared as shown below in any of Schemes 1 through 7:

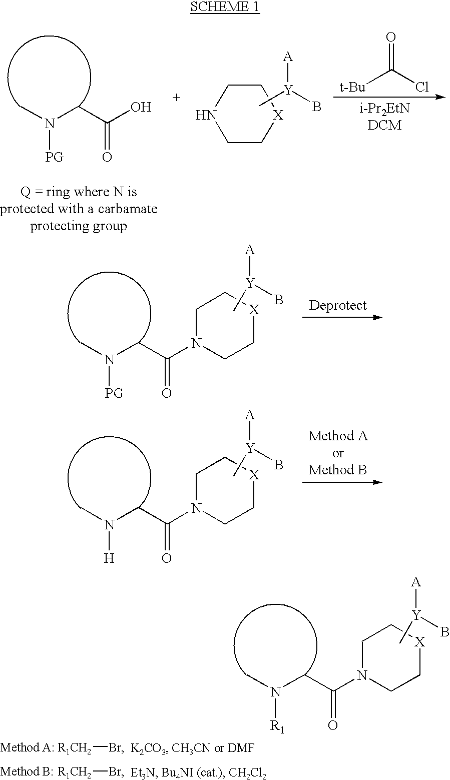

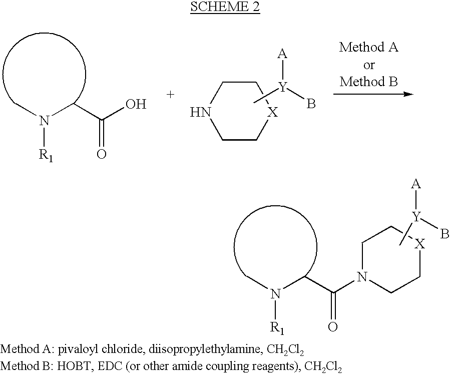

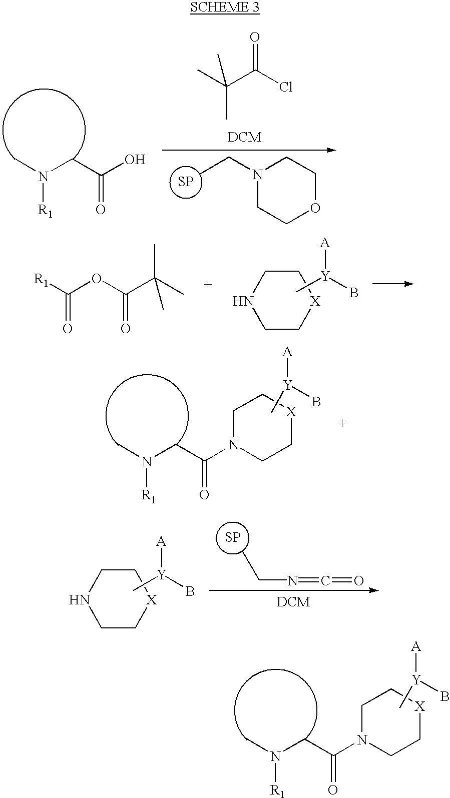

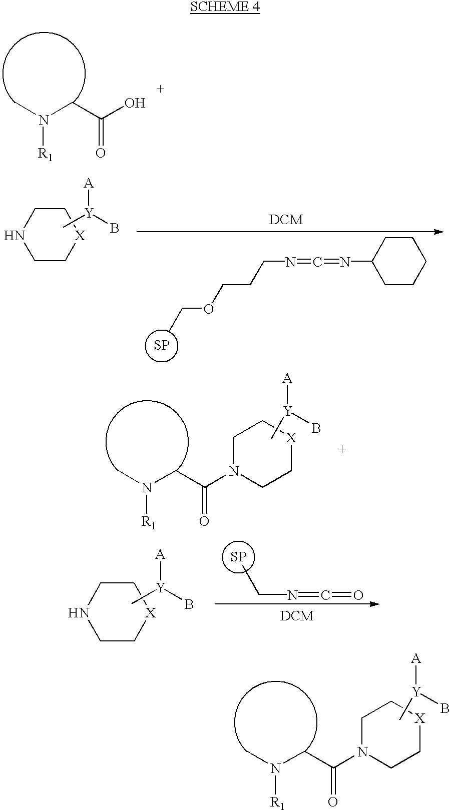

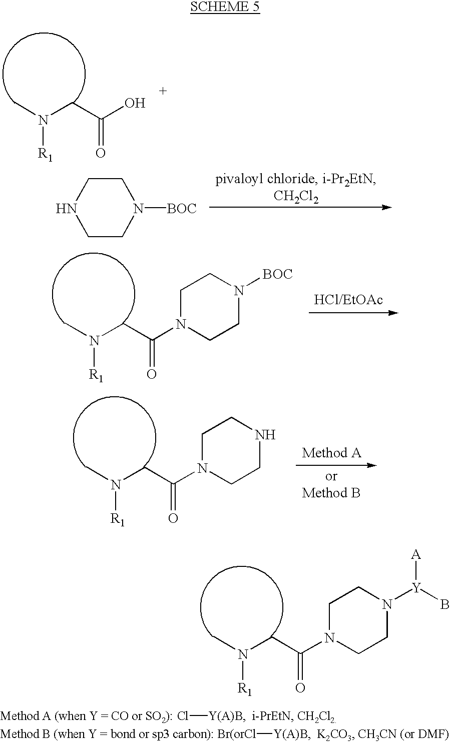

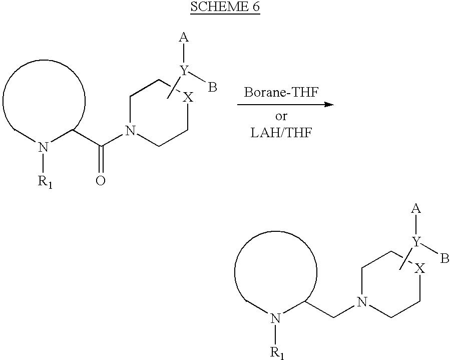

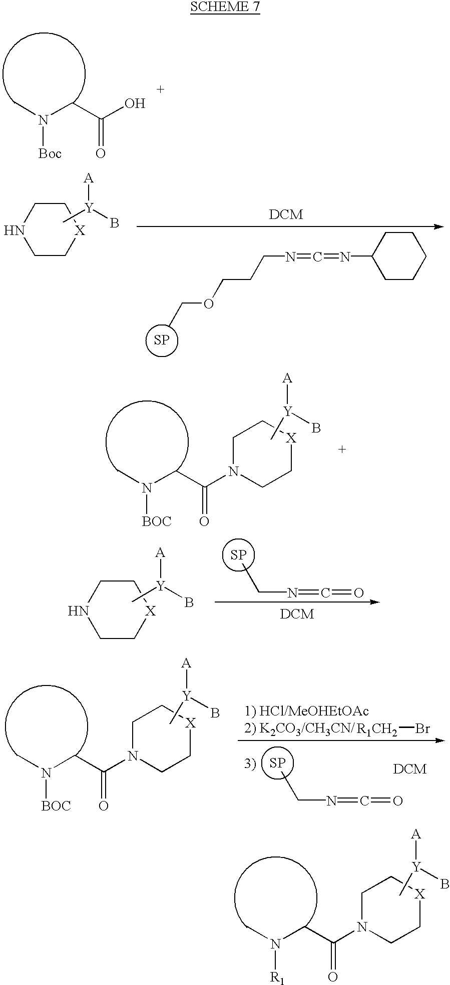

In the 7 schemes depicted above, the following abbreviations are used: tBu-C(O)-Cl=pivaloyl chloride; iPr₂EtN=diisopropylethylamine; DCM=dichloromethane; HCl=hydrogen chloride gas; EtOAc=ethyl acetate; Et₃N=triethylamine; DMF=dimethylformamide; THF=tetrahydrofuran; MeOH=methanol; Bu₄NI=tetrabutylammonium iodide; HOBT=N-hydroxybenzotriazole; EDC=1-(3-Dimethylaminopropyl)-3-ethylcarbodiimide hydrochloride; LAH=Lithium aluminum hydride. Schemes 3, 4 and 7 are combinatorial chemistry type wherein reactants linked to a polystyrene solid support (“SP”) are used.

Each of these schemes are described in more detail in the Example section.

One of skill in the art will be well aware of analogous synthetic methods for preparing compounds of formula (I).

Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxy methylcellulose, polyacrylates, waxes, polyethylene-polyoxypropylene-block polymers, polyethylene glycol and wool fat.

As used herein, the described compounds used in the pharmaceutical compositions and methods of this invention, are defined to include pharmaceutically acceptable derivatives thereof. A “pharmaceutically acceptable derivative” denotes any pharmaceutically acceptable salt, ester, or salt of such ester, of a compound of this invention or any other compound which, upon administration to a patient, is capable of modulating cholesterol biosynthesis in a mammal. If pharmaceutically acceptable salts of the described compounds are used, those salts are preferably derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The described compounds utilized in the methods of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The methods of the present invention used to modulate the cholesterol biosynthesis are also useful in treating diseases mediated by the cholesterol biosynthesis. According to the present invention, the term “diseases mediated by cholesterol biosynthesis” means any condition that either manifests or is characterized by an enhanced or decreased level of cholesterol. One of skill in the art will readily appreciate that the methods of the present invention can be selectively used to either enhance or decrease the cholesterol biosynthesis. This selectivity is derived by the ability of the compounds used in the present invention to either up-regulate or down-regulate the transcription levels of the genes involved in cholesterol biosynthesis.

The methods of the present invention can be used to treat Creutzfeld-Jakob disease, including the sporadic, inherited and the infectious forms, bovine spongiform encephalopathy, scrapie, Niemann-Pick Type C disease, Smith-Lemli-Opitz syndrome-and Tangier disease.

In a preferred embodiment, the methods are used to treat Creutzfeldt-Jakob disease, including the sporadic, inherited and the infectious forms, bovine spongiform encephalopathy and scrapie.

In a particularly preferred embodiment, the methods are used to treat Creutzfeldt-Jakob disease, including the sporadic, inherited and the infectious forms and bovine spongiform encephalopathy.

The amount of a described compound that may be combined with the carrier materials to produce a single dosage form will vary depending upon the host treated and the particular mode of administration. Preferably, the compositions should be formulated so that a dosage of between 0.01-100 mg/kg body weight/day of the described compound can be administered.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular described compound.

For use of the compounds according to the invention as medications, they are administered in the form of a pharmaceutical preparation containing not only the active ingredient but also carriers, auxiliary substances, and/or additives suitable for enteric or parenteral administration. Administration can be oral or sublingual as a solid in the form of capsules or tablets, as a liquid in the form of solutions, suspensions, elixirs, aerosols or emulsions, or rectal in the form of suppositories, or in the form of solutions for injection which can be given subcutaneously, intramuscularly, or intravenously, or which can be given topically or intrathecally. Auxiliary substances for the desired medicinal formulation include the inert organic and inorganic carriers known to those skilled in the art, such as water, gelatin, gum arabic, lactose, starches, magnesium stearate, talc, vegetable oils, polyalkylene glycols, etc. The medicinal formulations may also contain preservatives, stabilizers, wetting agents, emulsifiers, or salts to change the osmotic pressure or as buffers.

Solutions or suspensions for injection are suitable for parenteral administration, and especially aqueous solutions of the active compounds in polyhydroxy-ethoxylated castor oil.

Surface-active auxiliary substances such as salts of gallic acid, animal or vegetable phospholipids, or mixtures of them, and liposomes or their components, can be used as carrier systems.

In order that this invention be more fully understood, the following examples are set forth. These examples are for the purpose of illustration only and are not to be construed as limiting the scope of the invention in any way.

### EXAMPLE 1

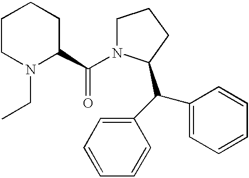

1-[(S)-2-(1,1-Diphenylmethyl)-pyrrolidin-1-yl]-1-((S)-1-ethyl-piperidin-2-yl)-methanone (Compound 27)

To a solution of 1-ethyl-(2S)-piperidine-2-carboxylic acid (158 mg, 11.0 mmols, 1.2 eq.) in 5 mL anhydrous DCM was added N,N-diisopropyl-ethylamine (585 μL, 3.4 mmols, 4.0 eq.) The reaction was stirred under N₂ for 10 min. then treated with pivaloyl chloride (124 μL, 11.0 mmols, 1.2 eq.) drop-wise via syringe. The reaction was stirred 1.5 h, then treated with a solution of (S)-2-(1,1-diphenyl-methyl)-pyrrolindine (0.199 mg, 0.84 mmols, 1.0 eq.) in 2 mL anhydrous DCM drop-wise, and stirred at room temperature (“RT”) for 96 h. The reaction was diluted with 20 mL DCM and washed with 20 mL saturated NaHCO₃. The aqueous layer was extracted twice with 20 mL DCM, then the combined organics were washed with water and brine, dried over sodium sulfate, filtered, and evaporated. The residue was purified via flash chromatography (98/2 dichloromethane/methanol) yielding 261 mg product. The product was then dissolved in 20 mL DCM and washed twice with saturated NaHCO₃. The basic layer was extracted once with 20 mL DCM, and the combined organics were washed once with water and once with brine, dried over sodium sulfate, filtered, and evaporated in vacuo to afford 172 mg (58%) of the title compound. ¹H NMR (Bruker, 500 MHz, CD₃OD): δ 7.50-7.10 (m, 10H), 5.25 (m, 1H), 4.50-4.10 (dd, rotomers, 1H), 4.00-3.65 (m, 1H), 3.60-3.30 (m, 1H), 3.20-2.80 (m, 2H), 2.70-2.50 & 2.30-2.15 (m, rotomers, 1H); 2.10-1.20 (m, 12H). 1.10 & 0.90 (t, rotomers, 3H) ppm. MS (M+H) 377.

Using the procedure described in Example 1 the compounds set forth in Examples 2 through 8 were prepared:

### EXAMPLE 2

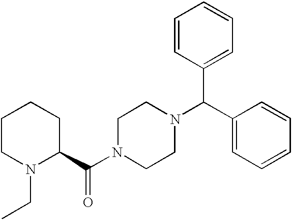

1-[4-(1,1-Diphenylmethyl)piperazin-1-yl]-1-((S)-1-ethylpiperidin-2-yl)methanone (Compound 1)

¹H NMR (CDCl₃, 500 MHz) δ 7.35(m, 4H), 7.18 (m, 4H), 7.11 (m, 2H), 4.16 (s, 1H, Ph2CH), 3.99 (br s, 1H), 3.78 (br. s, 1H), 3.61 (br. s, 1H), 3.51 (br. s, 1H), 2.98 (m, 2H), 2.55 (m, 1H), 2.32-2.22 (m, 4H), 2.10 (m, 1H), 1.78 (m, 1H), 1.62-1.38 (m, 5H), 1.18 (m, 1H), 0.95 (t, 3H) ppm.

MS (M+H): 392.5

### EXAMPLE 3

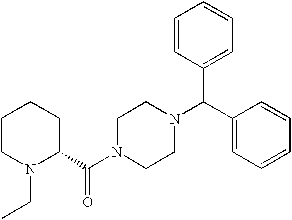

1-[4-(1,1-Diphenylmethyl)piperazin-1-yl]-1-((R)-1-ethylpiperidin-2-yl)methanone (Compound 15)

¹H NMR (CDCl₃, 500 MHz) δ 7.35 (m, 4H), 7.2 (m, 4H), 7.08 (m, 2H), 4.12 (s, 1H), 3.98 (br. s, 1H), 3.78 (br. s, 1H), 3.59 (br. s, 1H), 3.51 (br. s, 1H), 2.98 (m, 2H), 2.58 (m, 1H), 2.35-2.25 (m, 4H), 2.10 (m, 1H), 1.81 (m, 1H), 1.65-1.40 (m, 5H), 1.14 (m, 1H), 0.95 (t, 3H) ppm.

MS (M+H): 392.5

### EXAMPLE 4

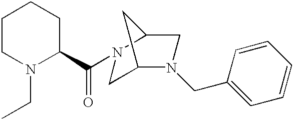

1-(5-benzyl-2,5-diaza-bicyclo[2.2.1]-hept-2-yl)-1-((S)-1-ethyl-piperidino-2-yl)-methanone (Compound 24)

82 mg (33%) crystalline product. ¹H NMR (Bruker 500 MHz, CD₃OD): δ 1.1 (m, 3H); 1.2-1.5 (m, 2H); 1.5-1.9 (m, 6H); 2.0-2.4 (m, 3H); 2.6-2.8 (m, 2H) 2.9 (m, 1H); 3.1-3.3 (m, 2H); 3.4-3.65 (m, 2H); 3.7-3.9 (m, 2H); 4.6-4.8 (dd, 1H); 7.2 (t, 1H); 7.3-7.4 (m, 4H) ppm.

MS (M+H): 377

### EXAMPLE 5

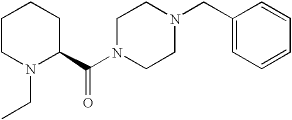

1-(4-Benzylpiperazin-1-yl)-1-((S)-1-ethylpiperidin-2-yl)methanone (Compound 16).

¹H NMR (CDCl₃, 500 MHz) δ 7.25 (m, 4H), 7.21 (m, 1H), 3.95 (br.s, 1H), 3.74 (br. s, 1H), 3.61 (br. s, 1H), 3.52 (br. s, 1H), 3.41 (s, 2H, PhCH₂), 3.04 (m, 2H), 2.58 (m, 2H), 2.36-2.28 (m, 4H), 2.12 (m, 1H), 1.80 (m, 1H), 1.71-1.42 (m, 4H), 1.18 (m, 1H), 0.95 (t, 3H) ppm. MS (M+H): 316.4

### EXAMPLE 6

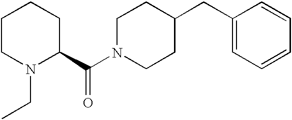

1-(4-Benzylpiperidin-1-yl)-1-((S)-1-ethylpiperidin-2-yl)methanone (Compound 26).

¹H NMR (CDCl₃, 500 MHz) δ 7.25-7.05 (m, 5H), 4.65 (br.s, 1H), 4.54 (d, 2H), 3.07 (m, 2H), 2.82 (m, 1H), 2.58-2.38 (m, 4H), 2.12 (m, 1H), 1.82 (m, 1H), 1.7-1.38 (m, 7H), 1.22 (m, 1H), 1.06 (m, 2H), 0.96 (t, 3H) ppm. MS (M+H): 315.4

### EXAMPLE 7

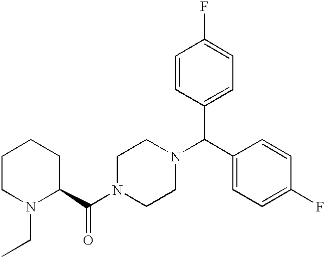

1-{4-[1,1-Bis-(4-fluorophenyl)methyl]-piperazin-1-yl}-1-((S)-1-ethylpiperidin-2-yl)methanone (Compound 25).

¹H NMR (CDCl₃, 500 MHz) δ 7.53 (m, 4H), 7.14 (m, 4H), 4.39 (s, 1H), 4.22 (br. s, 1H), 3.98 (br. s, 1H), 3.84 (br. s, 1H), 3.74 (br. s, 1H), 3.25 (m, 2H), 2.81 (m, 1H), 2.54 (m, 2H), 2.48 (m, 2H), 2.36 (m, 1H), 2.04 (m, 1H), 1.90 (m, 2H), 1.84-1.62 (m, 3H), 1.41 (m, 1H), 1.18 (t, 3H) ppm.

### EXAMPLE 8

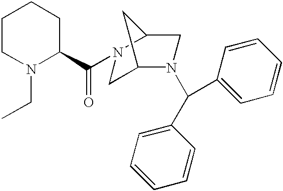

1-[(1S, 4S)-5-(1,1-Diphenylmethyl)-5-diazabicyclo[2.2.1]-hept-2-yl]-1-((S)-1-ethylpiperidin-2-yl)methanone (Compound 17).

¹H NMR (CDCl₃, 500 MHz) δ 7.38 (m, 4H), 7.18 (m, 4H), 7.12 (m, 2H), 4.76 (s, 0.5H), 4.52 (s, 1H), 4.43 (s, 0.5H), 3.65 (m, 1H), 3.38 (m, 1H), 3.22-2.98 (m, 2H), 2.85-2.46 (m, 3H), 2.33 (m, 1H), 2.08 (m, 1H), 1.92-1.10 (m, 9H), 1.02 & 0.97 (two t, 3H) ppm.

### EXAMPLE 9

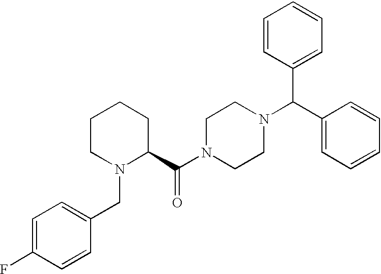

1-[4-(1,1-Diphenyl-methyl)-piperazin-1-yl]-1-[(S)-1-(4-fluoro-benzyl)-piperidin-2-yl]-methanone (Compound 21).

To a solution of 120 mg of 1-[4-(1,1-Diphenylmethyl)-piperazin-1-yl]-1-(S)-piperidin-2-yl-methanone dihydrochloride (0.28 mmol, 1 equiv.) in 10 mL of acetonitrile was added 300 mg of potassium carbonate (2.17 mmol, 8 equiv.) and 200 μL of 4-flourobenzyl bromide (1.6 mmol, 6 equiv.). The reaction was allowed to stir at 25° C. for 1 hr and then concentrated to a white solid which was extracted with dichloromethane and concentrated to a pale yellow oil. The crude product was purified by silica gel chromatography (20:1 methylene chloride:methanol, R_{f}=0.2), yielding 56 mg (0.118 mmol, 42% yield) of 1-[4-(1,1-Diphenyl-methyl)-piperazin-1-yl]-1-[(S)-1-(4-fluoro-benzyl)-piperidin-2-yl]-methanone as a clear oil. ¹H NMR (CDCl₃, 500 MHz) δ 7.35-7.05 (10H, m, Ar), 6.90-6.75 (4H, m, Ar), 4.05 (1H, s, Ph₂CH), 3.7 (1H, d, m, ArCH₂), 3.5 (1H, br s), 3.1 (1H, m), 2.2 (4H, br s), 1.5 (4H, br s), 1.35 (3H, br s), 1.1 (2H, br s) ppm. MS: 472.44(M+H) found.

### EXAMPLE 10

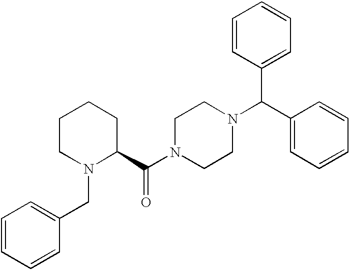

1-((S)-1-Benzyl-piperidin-2-yl)-1-[4-(1,1-diphenyl-methyl)-piperazin-1-yl]-methanone (Compound 20).

Compound 20 was prepared similarly to Compound 21, above, in Example 9.

¹H NMR (CDCl₃, 500 MHz) δ 7.35-7.05 (15H, m, Ar), 4.10 (1H, s, PH₂CH), 3.8 (1H, d, m, ArCH₂), 3.5 (3H, br s), 3.1 (1H, m), 2.85 (1H, br s), 2.2 (4H, br s), 1.5 (4H, br s), 1.35 (3H, br s), 1.1 (2H, br s) ppm. MS: 454.47 (M+H) found.

### EXAMPLE 11

Combinatorial Synthesis of Compounds Via Scheme 3

To N-ethylpipecolinic acid (0.157 g, 1.0 mmol) in 14 mL of dry CH₂Cl₂ was added pivaloyl chloride (0.121 g, 1.01 mmol) neat. After 1 hr, 1 mL of the resulting reaction solution was added to 14 wells of a reaction block containing morpholinomethyl polystyrene HL resin (100 mg, 0.4 mmol) and the appropriate amine derivative (0.2 mmol) in 2 mL of dry CH₂Cl₂. After shaking for 12 hrs, polystyrene methyl isocyanate (80 mg, 0.1 mmol) was added and the reaction solution was shaken an additional 12 hrs. Filtration and evaporation afforded the crude amide derivatives. Purification was accomplished with solid phase extraction (SPE-C) with methanol and methanol/ammonia to give the desired product.

Compounds 1 and 2 were synthesized in this manner.

### EXAMPLE 12

Combinatorial Synthesis of Compounds Via Scheme 4

To N-cyclohexanecarbodiimide-N′-propyloxymethyl polystyrene resin (150 mg, 0.15 mmol) in the wells of a reaction block was added the appropriate carboxylic acid derivative (0.075 mmol) neat. To each well was added 3 ml of 1-benzhydrylpiperazine (0.05 mmol) in dry CH₂Cl₂. After shaking for 12 hrs, polystyrene methyl isocyanate (80 mg, 0.1 mmol) was added and the reaction solution was shaken an additional 12 hrs. Filtration and evaporation afforded the crude amide derivatives. Purification was accomplished with reverse phase HPLC with H₂O/acetonitrile (0.1% TFA) to give the desired product as a trifluoroacetate salt.

Compounds 4, 7, 8 and 11 were synthesized in this manner.

### EXAMPLE 13

Synthesis of ((2S,4R)-1-Benzyl-4-hydroxypyrrolidin-2-yl)-(4-benzylpiperidin-1-yl)-methanone (Compound 30)

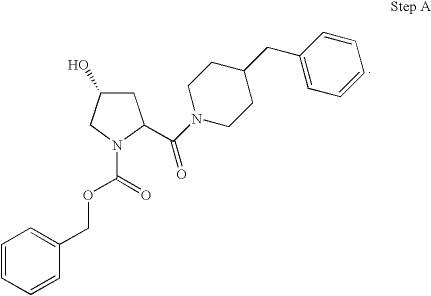

(2S,4R)-2-(4-Benzylpiperidine-1-carbonyl)-4-hydroxypyrrolidine-1-carboxylic Acid Benzyl Ester (Compound 32).

(2S,4R)-4-Hydroxy-pyrrolidine-1,2-dicarboxylic acid 1-benzyl ester (5.00 g, 19 mmol) was dissolved in 50 mL anhydrous dichloromethane and 11 mL (63 mmol) of N,N-diisopropylethylamine. Pivaloyl chloride (2.32 mL, 19 mmol) was added dropwise and the solution was stirred for 1 hour. Next, 4-benzylpiperidine (2.76 mL, 16 mmol) was added, and the solution was stirred for 16 hours. The reaction was diluted with dichloromethane, and then washed with saturated sodium bicarbonate, water, and brine. The organic layer was dried over sodium sulfate, filtered, and evaporated in vacuo to give a yellow oil that was purified by flash column chromatography (SiO₂) eluting with a gradient from ethyl acetate to dichloromethane to 2.0% methanol in dichloromethane. ¹H NMR (CDCl₃, 500 MHz): □ 0.8-1.3(m, 2H); 1.3-1.9 (m, 4H); 2.0-2.15 (m, 2H); 2.2 (m, 1H); 2.3-2.5 (m, 1H); 2.6 (m, 2H); 2.7-3.1 (4t, 1H); 3.6 (d, 0.5H); 3.7 (m, 0.5H); 3.8 (m, 1.5H); 4.0 (t, 0.5H); 4.4-4.7 (m, 2H); 5.0-5.3 (m, 2H); 7.0-7.4 (m, 10H) ppm. MS: m/z 423 (M+1).

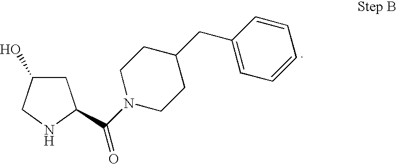

(4-Benzylpiperidin-1-yl)-((2S,4R)-4-hydroxypyrrolidin-2-yl)-methanone (Compound 33).

We dissolved (2S,4R)-2-(4-Benzyl-piperidine-1-carbonyl)-4-hydroxy-pyrrolidine-1-carboxylic acid benzyl ester (2.77 g, 6.5 mmol) in 50 mL anhydrous EtOH, and degassed with N₂. Add Pd(OH)₂ (1.7 g, cat.) and stir under H₂ (1 atm.). The reaction was filtered through Celite and evaporated to afford an orange foam (1.96 g, 100%). ¹H NMR (CDCl₃, 500 MHz): [symbol] 0.9-1.2 (m, 2H); 1.5-1.8 (m, 4H); 2.2 (m, 1H); 2.4 (m, 3H); 2.8 (q, 1H); 3.0 (dd, 1H); 3.1 (dd, 1H); 3.8 (d, 1H); 4.2 (t, 3H); 4.4 (d, 2H); 7.0 (d, 2H); 7.1 (t, 1H); 7.2 (m, 2H) ppm. MS: m/z 289 (M+1)

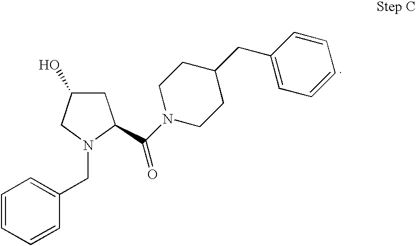

((2S,4R)-1-Benzyl-4-hydroxypyrrolidin-2-yl)-(4-benzylpiperidin-1-yl)-methanone (Compound 30).

(4-Benzylpiperidin-1-yl)-((2S,4R)-4-hydroxy-pyrrolidin-2-yl)-methanone (1.74 g, 6.0 mmol) was dissolved in 100 mL acetonitrile. Potassium carbonate (3.34 g, 24 mmol) was added to the solution followed by the addition of benzyl bromide (0.450 ml, 6.0 mmol). The mixture was stirred for 1 hour, filtered, and evaporated in vacuo to afford a viscous oil. The crude product was purified by flash chromatography (SiO₂) eluting with a gradient of EtOAc to 9:1 EtOAc/Methanol to give 1.13 g (50%) of the desired product. ¹H NMR (CDCl₃, 500 MHz): □1.0 (m, 2H); 1.45 (d, 0.5H); 1.5-1.7 (broad d, 2.5H); 1.7-2.0 (m, 2H); 2.0-2.1 (m, 0.5H); 2.1-2.2 (m, 0.5H); 2.3-2.6 (m, 4H); 2.6-2.8 (m, 1H); 3.4 (broad s, 1H); 3.5-3.7 (m, 1H); 3.8 (broad s, 2H); 3.9 (d, 1H); 4.4 (broad s, 1H); 4.5 (broad t, 1H); 7.0 (d, 2H); 7.1-7.3 (m, 8H) ppm. MS: m/z 379 (M+1).

### EXAMPLE 14

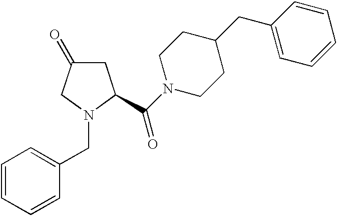

(2S)-1-Benzyl-5-(4-benzylpiperidine-1-carbonyl)-pyrrolidin-3-one (Compound 29).

Oxalyl chloride (0.065 ml, 0.72 mmol) was added dropwise to a cooled (−78° C.) solution of DMSO (0.10 ml, 1.37 mmol) in 10 mL of anhydrous dichloromethane. The mixture was stirred at −65° C. for 2 hours. ((2S,4R)-1-Benzyl-4-hydroxy-pyrrolidin-2-yl)-(4-benzyl-piperidin-1-yl)-methanone (140 mg, 0.37 mmol) in 5 mL anhydrous dichloromethane was added to the solution dropwise. After stirring for 2.5 hours at −45° C., N,N-diisopropylethylamine (0.35 ml, 2 mmol) was added dropwise. The reaction was warmed to 0° C. and diluted with dichloromethane. The reaction was washed with saturated sodium bicarbonate, water and brine. The organic layer was dried over sodium sulfate, filtered, and evaporated. The crude residue was purified by flash chromatography (SiO₂) using a gradient from dichloromethane to 2%MeOH in dichloromethane, yielding 101 mg (79%) of the desired product. ¹H NMR (CDCl₃, 500 MHz): □ 1.0 (m, 2H); 1.5-1.6 (m, 1H); 1.6-1.7 (m, 2H) 2.3-2.5 (m, 4H); 2.6 (d, 1H); 2.7-2.8 (m, 1H); 3.0 (d, 1H); 3.5 (t, 1H); 3.6-3.8 (m, 2H); 3.9 (br. s, 1H); 4.1 (br. s, 1H); 4.5 (br. s, 1H); 7.05 (t, 2H); 7.15 (m, 1H); 7.25 (m, 7H) ppm. MS: m/z 377 (M+1).

### EXAMPLE 15

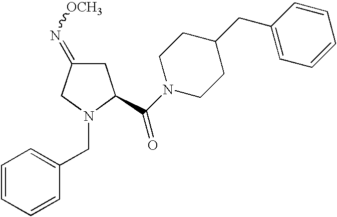

(2S)-1-Benzyl-5-(4-benzylpiperidine-1-carbonyl)-pyrrolidin-3-one O-methyl-oxime (Compound 31).

(S)-1-Benzyl-5-(4-benzyl-piperidine-1-carbonyl)-pyrrolidin-3-one (Compound 29) (70 mg, 0.19 mmol) and methoxylamine hydrochloride (20 mg, 0.25 mmol) were taken into 5 mL anhydrous methanol and heated to 40° C. for 2 hours. The reaction was evaporated and then partitioned between dichloromethane and saturated sodium bicarbonate. The aqueous layer was extracted with dichloromethane and the combined organic extracts were washed with brine, dried over sodium sulfate, filtered, and evaporated. The crude residue was purified by flash chromatography (SiO₂) using a gradient from 0%-2% methanol in dichloromethane to yield 25 mg (33%) of the desired product. ¹H NMR (CDCl₃, 500 MHz): [symbol] .8-1.2 (m, 3H); 1.4-1.8 (m, 3H); 2.3-2.5 (m, 3H); 2.5-2.7 (m, 1H); 2.7-2.9 (m, 2H); 3.1-3.3 (m, 1H); 3.45 (t, 0.5H); 3.6 (d, 0.5H); 3.6-3.8(m, 4H); 3.8-4.0 (m, 2H); 4.5 (br. s, 1H); 7.0 (d, 2H); 7.15 (m, 1H); 7.2-7.4 (m, 7H) ppm. MS: m/z 406 (M+1).

### EXAMPLE 16

The compounds described in Examples 16-32 were prepared by the procedure described in Example 1 (Scheme 2).

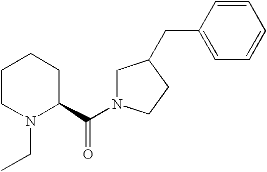

(3-Benzylpyrrolidin-1-yl)-((2S)-1-ethylpiperidin-2-yl)-methanone (Compound 35).

Compound 35 was prepared from (2S)-1-ethylpiperidin-2-yl carboxylic acid and 3-benzylpyrrolidine as described in Example 1 to yield 62 mg (17%). ¹H NMR (CDCl₃, 500 MHz): [symbol] 1.35 (m, 3H); 1.4-1.7 (m, 3H); 1.8 (m, 2H); 2.0-2.2 (m, 2H); 2.3-2.7 (m, 4H); 2.9-3.25 (m, 4H); 3.25-3.7 (m, 3H); 4.0 (bs, 1H); 4.1-4.2 (m, 1H); 7.1 (m, 2H); 7.1-7.3 (m, 3H) ppm.

### EXAMPLE 17

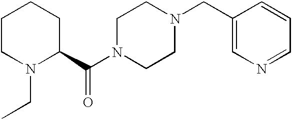

((2S)-1-Ethylpiperidin-2-yl)-(4-pyridin-3-ylmethylpiperazin-1-yl)-methanone Trihydrochloride (Compound 36).

Compound 36 was prepared from (2S)-1-ethylpiperidin-2-yl carboxylic acid and 3-pyridinylmethylpiperazine as described in Example 1 to afford 229 mg (72%) as the trihydrochloride salt. ¹H NMR (CDCl₃, 500 MHz): [symbol] 1.0 (t, 3H); 1.2 (m, 1H); 1.4-1.8 (m, 5H); 1.85 (bs, 1H); 2.15 (bs, 1H); 2.15 (bs, 1H); 2.4 (m, 4H); 2.6 (bs, 1H); 3.1 (bs, 2H); 3.4 (s, 2H); 3.5 (bs, 1H); 3.6 (s, 1H); 3.8 (bs, 1H); 4.0 (bs, 1H); 7.2 (d, 1H); 7.6 (d, 1H); 8.5 (m, 2H) ppm. MS: m/z 317 (M+1).

### EXAMPLE 18

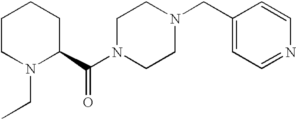

((2S)-1-Ethylpiperidin-2-yl)-(4-pyridin-4-ylmethylpiperazin-1-yl)-methanone Trihydrochloride (Compound 37).

Compound 37 was prepared from (2S)-1-ethylpiperidin-2-yl carboxylic acid and 4-pyridinylmethylpiperazine as described in Example 1 to yield 236 mg (75%) as the trihydrochloride salt. ¹H NMR (CDCl₃, 500 MHz): [symbol] 1.0 (t, 3H); 1.2 (m, 1H); 1.4-1.8 (m, 5H); 1.85 (bs, 1H); 2.15 (bs, 1H); 2.15 (bs, 1H); 2.4 (m, 4H); 2.6 (bs, 1H); 3.1 (bs, 2H); 3.4 (s, 2H); 3.5 (bs, 1H); 3.6 (s, 1H); 3.8 (bs, 1H); 4.0 (bs, 1H); 7.2 (d, 2H); 8.5 (d, 2H) ppm. MS: m/z 317 (M+1).

### EXAMPLE 19

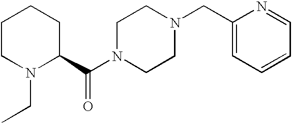

((2S)-1-Ethylpiperidin-2-yl)-(4-pyridin-2-ylmethylpiperazin-1-yl)-methanone Trihydrochloride (Compound 38).

Compound 38 was prepared from (2S)-1-ethylpiperidin-2-yl carboxylic acid and 2-pyridinylmethylpiperazine as described in Example 1 to yield 42 mg (13%) as the trihydrochloride salt. ¹H NMR (CDCl₃, 500 MHz): [symbol] 1.0 (t, 3H); 1.2 (s, 3H); 1.4-1.8 (m, 2H); 1.85 (m, 1H); 2,1 (m, ₁₁H); 2.4 (m, 4H); 2.6 (bs, 1H); 3.0 (bs, 2H); 3.6 (s, 5H); 3.8 (bs, 1H); 4.0 (bs, 1H); 7.1 (t, 1H); 7.3 (d, 1H); 7.6 (t, 1H); 8.5 (d, 1H) ppm. MS: m/z 317 (M+1).

### EXAMPLE 20

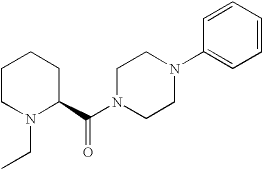

((2S)-1-Ethylpiperidin-2-yl)-(4-phenylpiperazin-1-yl)-methanone Dihydrochloride (Compound 39).

Compound 39 was prepared from (2S)-1-ethylpiperidin-2-yl carboxylic acid and N-phenylpiperazine as described in Example 1 to yield 277 mg (74%) as the dihydrochloride salt. ¹H NMR (CDCl₃, 500 MHz): [symbol] 1.3 (t, 3H); 1.6 (m, 1H); 1.7 (q, 2H); 1.9 (m, 2H); 2.1 (d, 1H); 3.0 (2H); 3.2 (m, 1H); 3.5 (m, 4H); 3.7 (d, 1H); 3.9 (m, 4H); 4.4 (m, 1H); 7.3 (m, 3H); 7.5 (m, 2H). MS: m/z 406 (M+1) ppm.

### EXAMPLE 21

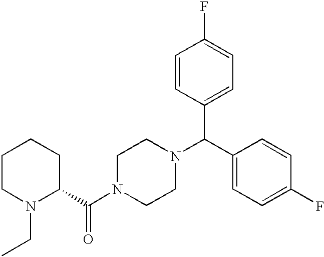

{4-[Bis-(4-fluorophenyl)methyl]-piperazin-1-yl}-((2R)-1-ethylpiperidin-2-yl)-methanone (Compound 40).

Compound 40 was prepared from (2R)-1-ethylpiperidin-2-yl carboxylic acid and N-Bis-(4-fluorophenyl)methylpiperazine as described in Example 1 to yield 590 mg (46% yield) after chromatography. ¹H NMR (500 MHz, CDCl₃), δ 7.40-7.35 (m, 4H), 7.05-6.95 (m, 4H), 4.20 (s, 1H), 4.05-3.50 (m, 4H), 3.10-3.00 (m, 2H), 2.40-2.25 (m, 4H), 1.85-1.40 (m, 8H), 1.35-1.00 (m, 4H) ppm. MS: m/z 428.5 (M+1).

### EXAMPLE 22

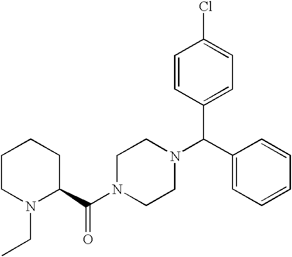

{4-[(4-Chlorophenyl)phenylmethyl]-piperazin-1-yl}-((2S)-1-ethylpiperidin-2-yl)-methanone Dihydrochloride (Compound 41).

Compound 41 was prepared from (2S)-1-ethylpiperidin-2-yl carboxylic acid and N-(4-chlorophenyl) phenylmethylpiperazine as described in Example 1 to yield 170 mg (67%) as the dihydrochloride salt. ¹H NMR (CDCl₃, 500 MHz) δ 7.30 (m, 4H), 7.18 (m, 4H), 7.12 (m, 1H), 4.14 (s, 1H), 3.98 (m, 1H), 3.76 (m, 1H), 3.58 (m, 1H), 3.52 (m, 1H), 3.0 (m, 2H), 2.55 (m, 1H), 2.26 (m, 3H), 2.14 (m, 1H), 1.85 (m, 1H), 1.7 (m, 2H), 1.52 (m, 3H), 1.14 (m, 2H), 0.95 (m, 3H) ppm. MS: m/z 426.5 (M+1)

### EXAMPLE 23

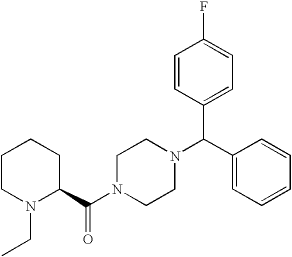

((2S)-1-Ethylpiperidin-2-yl)-{4-[(4-fluorophenyl)phenylmethy]-piperazin-1-yl}-methanone Dihydrochloride (Compound 42).

Compound 42 was prepared from (2S)-1-ethylpiperidin-2-yl carboxylic acid and N-(4-fluorophenyl)phenylmethylpiperazine as described in Example 1 to yield 282 mg (60%) as the dihydrochloride salt. ¹H NMR (DMSO-d₆, 500 MHz) δ 7.98 (m, 4H), 7.46 (m, 2H), 7.41 (m, 1H), 7.33 (m, 2H), 5.75 (m, 1H), 4.52-3.88 (m, 5H), 3.55 (m, 2H), 3.3-2.8 (m, 6H), 2.05 (m, 1H), 1.85 (m, 3H), 1.56 (m, 2H), 1.22 (t, 3H) ppm. MS m/z 410.5 (M+1).

### EXAMPLE 24

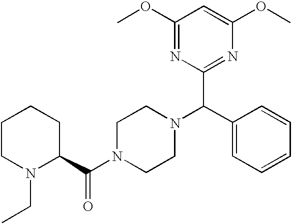

{4-[4,6-Dimethoxypyrimidin-2-yl)-phenylmethyl]-piperazin-1-yl}-((2S)-1-ethyl-piperidin-2-yl)-methanone (Compound 43).

Compound 43 was prepared from (2S)-1-ethylpiperidin-2-yl carboxylic acid and N-(4,6-dimethoxypyrimidin-2-yl)phenylmethylpiperazine as described in Example 1 to yield 184 mg (40%). ¹H NMR (CDCl₃, 500 MHz) δ 7.6 (m, 2H), 7.28 (m, 3H), 5.88 (s, 1H), 4.48 (m, 1H), 4.04 (m, 1H), 3.94 (s, 6H), 3.85 (m, 1H), 3.65 (m, 2H), 3.09 (m, 2H), 2.55 (m, 3H), 2.4 (m, 2H), 2.2 (m, 1H), 1.9 (m, 1H), 1.8-1.5 (m, 4H), 1.26 (m, 2H), 1.04 (m, 3H) ppm. MS m/z 454.4 (M+1).

### EXAMPLE 25

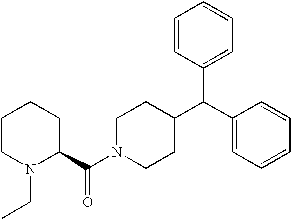

(4-Benzhydrylpiperidin-1-yl)-((2S)-1-ethylpiperidin-2-yl)-methanone Hydrochloride (Compound 44).

Compound 44 was prepared from (2S)-1-ethylpiperidin-2-yl carboxylic acid and 4-benzhydrylpiperidine as described in Example 1 to yield 174 mg (57%) as the hydrochloride salt. ¹H NMR (CDCl₃, 500 MHz) δ 7.24 (m, 8H), 7.12 (m, 2H), 4.28 (m, 1H), 4.38 (s, 1H), 3.99 (m, 1H), 3.63 (m, 1H), 3.42 (d, 1H), 3.20 (m, 3H), 3.00 (m, 1H), 2.53 (m, 2H), 2.32 (m, 1H), 2.15 (m, 1H), 1.82-1.60 (m, 5H), 1.50 (m, 1H), 1.35 (m, 3H), 1.03 (m, 2H) ppm. MS: m/z 391.5 (M+1).

### EXAMPLE 26

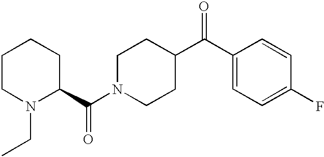

((2S)-1-Ethylpiperidin-2-yl)-[4-(4-fluorobenzoyl)-piperidin-1-yl]-methanone (Compound 45).

Compound 45 was prepared from (2S)-1-ethylpiperidin-2-yl carboxylic acid and 4-(4-fluorobenzoyl)piperidine as described in Example l to yield 830 mg (80%). ¹H NMR (CDCl₃, 500 MHz) δ 7.88 (m, 2H), 7.06 (m, 2H), 4.55 (m, 1H), 3.37 (m, 1H), 3.08 (m, 3H), 2.72 (m, 1H), 2.54 (m, 1H), 2.1 (m, 1H), 1.88-1.4 (m, 10H), 1.16 (m, 2H), 0.94 (m, 3H) ppm. MS m/z 347.3 (M+1).

### EXAMPLE 27

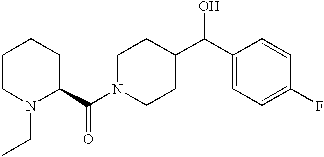

((2S)-1-Ethylpiperidin-2-yl)-{4-[(4-fluorophenyl)hydroxymethyl]-piperidin-1-yl}-methanone (Compound 46).

(2S)-1-Ethyl-piperidin-2-yl)-[4-(4-fluoro-benzoyl)-piperidin-1-yl]-methanone (Compound 45) (157 mg) was dissolved in 5 ml of ethanol. To the solution was added 50 mg of 10% palladium on carbon and the flask was charged with hydrogen (1 atm.). After stirring overnight, the reaction was filtered through Celite and the reaction evaporated in vacuo. The reaction was purified by flash chromatography (SiO₂) eluting with 95:5 dichloromethane/methanol to afford 95 mg of compound 46.

¹H NMR (DMSO-d₆, 500 MHz) δ 7.2 (m, 2H), 7.03 (m, 2H), 5.2 (br s, 1H), 4.43 (m, 1H), 4.17 (m, 2H), 3.78 (m, 1H), 3.37 (m, 1H), 3.81 (m, 4H), 2.42 (m, 1H), 1.8-1.5 (m, 6H), 1.42 (m, 2H), 1.06-0.92 (m, 5H) ppm. MS m/z 349.3 (M+1).

### EXAMPLE 28

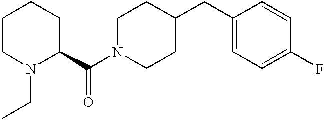

((2S)-1-Ethylpiperidin-2-yl)-[4-(4-fluorobenzyl)-piperidin-1-yl]-methanone hydrochloride (Compound 47).

Compound 47 was prepared from (2S)-1-ethylpiperidin-2-yl carboxylic acid and 4-(4-fluorobenzyl)piperidine as described in Example 1 to yield 379 mg (67%) as the HCl salt. ¹H NMR (CDCl₃, 500 MHz) δ 6.93 (m, 2H), 6.8 (m, 2H), 4.6 (m, 1H), 4.48 (m, 2H), 2.98 (m, 2H), 2.72 (m, 1H), 2.5 (m, 1H), 2.32 (m, 3H), 2.03 (m, 1H), 1.75 (m, 1H), 1.55 (m, 8H), 1.12 (m, 1H), 0.95 (m, 4H) ppm. MS m/z 333.4 (M+1).

### EXAMPLE 29

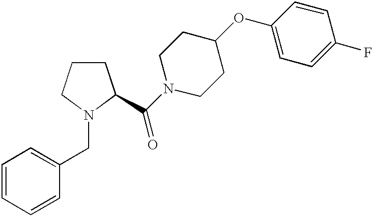

((2S)-1-Benzylpyrrolidin-2-yl)-[4-(4-fluorophenoxy)-piperidin-1-yl]-methanone Hydrochloride (Compound 48).

Compound 48 was prepared from (2S)-1-benzylpyrrolidin-2-yl carboxylic acid and 4-(4-fluorophenoxy)piperidine as described in Example 1 to yield 516 mg (65%) as the hydrochloride salt. ¹H NMR (CDCl₃, 500 MHz) δ 7.36 (m, 5H), 6.98 (m, 2H), 6.87 (m, 2H), 4.4 (m, 1H), 3.98 (m, 1H), 3.80 (m, 2H), 3.52 (m, 4H), 3.10 (m, 1H), 2.32 (m, 1H), 2.15 (m, 1H), 1.84 (m, 4H), 1.75 (m, 3H) ppm. MS: m/z 383.5 (M+1)

### EXAMPLE 30

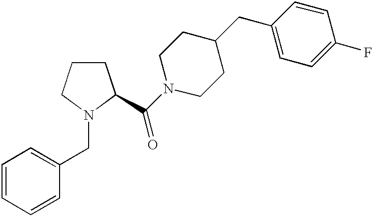

((2S)-1-Benzylpyrrolidin-2-yl)-[4-(4-fluorobenzyl)-piperidin-1-yl]-methanone Hydrochloride (Compound 49).

Compound 49 was prepared from (2S)-1-benzylpyrrolidin-2-yl carboxylic acid and 4-(4-fluorobenzyl)piperidine as described in Example 1 to yield 674 mg (81%) as the hydrochloride salt. ¹H NMR (CDCl₃, 500 MHz) δ 7.83 (m, 1H), 7.71 (m, 1H), 7.42 (m, 3H), 7.07 (m, 4H), 4.58 (m, 2H), 4.38 (m, 0.5H), 4.28 (m, 0.5H), 3.87-3.58 (m, 2H), 3.35 (m, 1H), 2.80 (m, 0.5H), 2.70 (m, 0.5H), 2.58-2.17 (m, 5H), 1.95 (m, 1H), 1.68 (m, 4H), 1.41 (m, 1H), 1.03 (m, 2H) ppm. MS: m/z 381.5 (M+1)

### EXAMPLE 31

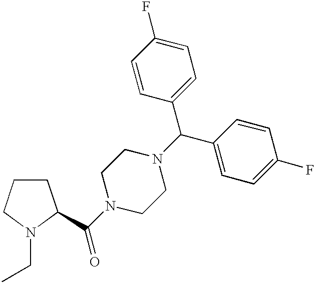

4-[Bis-(4-fluorophenyl)methyl]piperazin-1-yl}-((2S)-1-ethylpyrrolidin-2-yl)-methanone dihydrochloride (Compound 50)

Compound 50 was prepared from (2S)-1-ethylpyrrolidin-2-yl carboxylic acid and N-Bis-(4-fluorophenyl)methylpiperazine as described in Example 1 o yield 1.58 g (52%) as the dihydrochloride salt. ¹H NMR (CDCl₃, 500 MHz) δ 7.41 (m, 4H), 7.06 (m, 6H), 4.28 (s, 1H), 3.79 (m, 1H), 3.72 (m, 1H), 3.58 (m, 2H), 3.38 (m, 1H), 3.26 (m, 1H), 2.80 (m, 1H), 2.5-2.25 (m, 6H), 2.14 (m, 1H), 1.94 (m, 1H), 1.84 (m, 2H), 1.13 (t, 3H) ppm. MS: m/z 414.5 (M+1).

### EXAMPLE 32

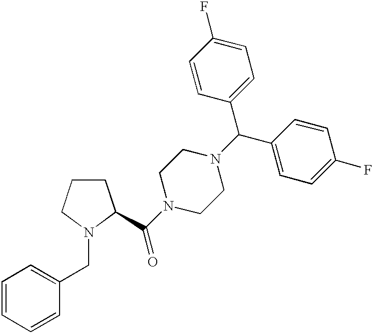

((2S)-1-Benzylpyrrolidin-2-yl)-{4-bis-(4-fluorophenyl)methyl]-piperazin-1-yl}-methanone dihydrochloride (Compound 51)

Compound 51 was prepared from (2S)-1-benzylpyrrolidin-2-yl carboxylic acid and N-Bis-(4-fluorophenyl)methylpiperazine as described in Example 1 to yield 1.59 g (66%) as the dihydrochloride salt. ¹H NMR (CDCl₃, 500 MHz) δ 7.4 (m, 2H), 7.02 (m, 2H), 4.26 (s, 1H), 3.92 (m, 1H), 3.69-3.4 (m, 3H), 3.32 (m, 1H), 2.39 (m, 3H), 1.65 (m, 3H), 1.45 (m, 6H) ppm. MS: m/z 476.5 (M+1).

The compounds described in Examples 33-34 were prepared by Scheme 2 (Method B).

### EXAMPLE 33

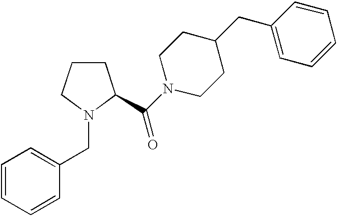

(4-Benzylpiperidin-1-yl)-((2S)-1-benzylpyrrolidin-2-yl)-methanone hydrochloride (Compound 52).

1-Benzyl-L-proline (3.12 g, 15 mmol)(was taken into 60 mL anhydrous dichloromethane. To this solution was added HOBT (2.06 g, 15 mmol), 4-benzylpiperidine (1.77 ml, 10.1 mmol), and EDC (3.84 g, 20 mmol). The reaction was stirred for 16 hours at room temperature. The reaction was diluted with dichloromethane, washed with saturated sodium bicarbonate, water and brine. The organic layer was dried over anhydrous sodium sulfate, filtered, and evaporated in vacuo. The crude residue was purified by flash chromatography (SiO₂) using 4% MeOH in dichloromethane yielding 3.60 g (98%) of compound 52 which was converted to the hydrochloride salt (3.41 g; 95%). ¹H NMR (D₂O, 500 MHz): [symbol] 0.4-1.0 (m, 2H); 1.4-1.6 (m, 2H); 1.7 (m, 1H); 1.8 (m, 1H); 1.9 (m, 1H); 2.1 (m, 1H); 2.4 (m, 4H); 2.8 (m, 1H); 3.3 (m, 1H); 3.5 (1H); 3.7-3.9 (m, 2H); 4.1 (dd, 1H); 4.5 (m, 1H); 4.4, 4.6 (dd, 1H); 7.1-7.4 (m, 10H) ppm. MS m/z 363 (M+1).

### EXAMPLE 34

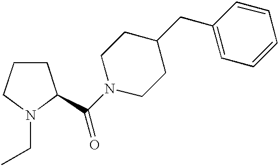

(4-Benzylpiperidin-1-yl)-((2S)-1-ethylpyrrolidin-2-yl)-methanone Hydrochloride (Compound 53).

Compound 53 was prepared from (2S)-1-ethylpyrrolidin-2-yl carboxylic acid and 4-benzylpiperidine as described in Example 33 to yield 233 mg (53%) of as the HCl salt. ¹H NMR (CDCl₃, 500 MHz): □ 1.0 (q, 2H); 1.3 (m, 2H); 1.7 (m, 3H); 1.9 (m, 1H); 2.0-2.2 (m, 2H); 2.4 (m, 3H); 2.5 (m, 1H); 2.8 (t, 0.5H); 2.9 (t, 0.5H); 3.2-3.4 (m, 3H); 3.5 (m, 1H); 3.7 (t, 1H); 4.4 (m, 1H); 4.6 (m, 1H); 7.0 (d, 2H); 7.1 (t, 1H); 7.2 (t, 2H) ppm. MS m/z 301 (M+1).

The compounds described in Examples 35-45 were prepared by Scheme 1.

### EXAMPLE 35

Preparation of (4-Benzylpiperidin-1-yl)-((2R)-1-benzylpyrrolidin-2-yl)-methanone (Compound 55)

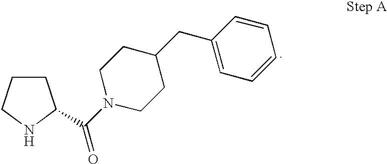

(4-Benzylpiperidin-1-yl-(2R)-pyrrolidin-2-yl-methanone Hydrochloride (Compound 54).

BOC-D-Proline (3.345 g, 15.5 mmol) was dissolved in 25 ml of dichloromethane. To the solution was added 4-benzylpiperidine (1.28 ml, 10.3 mmol), HOBT (2.1 g, 15.5 mmol), and EDC (3.96 g, 20.6 mmol). The reaction was stirred at room temperature for 16 hours. The reaction was diluted with 50 ml of dichloromethane and washed with saturated sodium bicarbonate, water, and brine. The organic layer was dried over anhydrous sodium sulfate, filtered, and evaporated to give a yellow oil that was purified by flash chromatography (SiO2) eluting with 95:5 dichloromethane/methanol to afford 3.22 g (58% yield) of (2R)-2-(4-Benzylpiperidine-1-carbonyl)-pyrrolidine-1-carboxylic acid tert-butyl ester. MS m/z 373 (M+1).

(2R)-2-(4-Benzylpiperidine-1-carbonyl)-pyrrolidine-1-carboxylic acid tert-butyl ester (3.22 g, 8.6 mmol) was dissolved in 50 ml of ethyl acetate. The solution was treated with anhydrous HCl and stirred at room temperature for 1 hour. The reaction was evaporated in vacuo and dried to afford 2.5 g (94% yield) of compound 54. ¹H NMR (CDCl₃, 500 MHz) δ 7.35 (m, 2H), 7.2 (m, 1H), 7.1 (d, 2H), 4.7 (br. s, 1H), 4.5 ((t, 1H), 3.7 (t, 1H), 3.6 (m, 1H), 3.4 (br. s, 1H), 3.1 (m, 1H), 2.7 (m, 1H), 2.6 (m, 2H), 2.5 (m, 1H), 2.2 (m, 1H), 2.1-2.0 (m, 1H), 1.9 (m, 1H), 1.85-1.70 (m, 3H), 1.6 (m, 1H), 1.4-1.1 (m, 2H) ppm. MS m/z 309 (M+1).

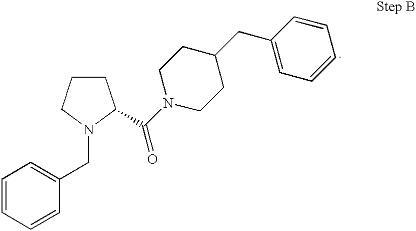

(4-Benzylpiperidin-1-yl)-((2R)-1-benzylpyrrolidin-2-yl)methanone Hydrochloride (Compound 55).

(4-Benzylpiperidin-1-yl-(2R)-pyrrolidin-2-yl-methanone hydrochloride (67 mg, 0.22 mmol) was dissolved in 5 ml of dichloromethane. To the solution was added benzyl bromide (25 μl, 0.22 mmol), triethylamine (60 μl, 0.44 mmol), and 5 mg of tetrabutylammonium iodide. The solution was stirred at room temperature for 16 hours. The reaction was diluted with 25 ml of dichloromethane and washed with saturated sodium bicarbonate, water, and brine. The organic layer was dried over anhydrous sodium sulfate, filtered, and evaporated in vacuo to afford a yellow oil. This was purified by flash chromatography (SiO₂) eluting with 100:2 dichloromethane/methanol to afford compound 55 which was converted to its hydrochloride salt, 43 mg (51% yield). 1H NMR ((D2O, 500 MHz) δ 7.3-7.1 (m, 8H), 7.05 (t, 2H), 4.50 (t, 1H), 4.10 (m, 1H), 3.90 (m, 1H), 3.40 (d, 0.5H), 3.30 (m, 1.5H), 3.0 (m, 1H), 2.75 (q, 1H), 2.5-2.3 (m, 3H), 2.2 (m, 1H), 2.0 (m, 1H), 1.85-1.45 (m, 7H), 1.1-0.85 (m, 2H) ppm.

### EXAMPLE 36

Preparation of (4-Benzylpiperidin-1-yl)-((2S)-1-phenethylpyrrolidin-2-yl)-methanone (Compound 57)

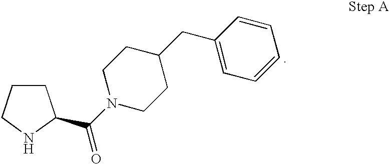

(4-Benzylpiperidin-1-yl)-(2S)-pyrrolidin-2-yl-methanone Hydrochloride (Compound 56).

2-(4-Benzylpiperidine-1-carbonyl)-pyrrolidine-1-carboxylic acid tert-butyl ester (10.4 g, 48 mmol) was subjected to identical conditions as the D isomer in Example 35, Step A (Compound 54) to yield 14.98 g (100%) of (2S)-2-(4-Benzylpiperidine-1-carbonyl)-pyrrolidine-1-carboxylic acid tert-butyl ester. MS m/z 373 (M+1).

The product, (2S)-2-(4-Benzylpiperidine-1-carbonyl)-pyrrolidine-1-carboxylic acid tert-butyl ester (14.98 g, 48 mmol, 1.2 equivalents) was dissolved in 150 mL EtOAc and HCl(g) was bubbled through for 15 min, then the reaction was stirred for 1 hour. The reaction was evaporated to afford 12.64 g (100%) of compound 56 as a white foam. ¹H NMR (CDCl₃, 500 MHz): [symbol] 1.1-1.4 (m, 2H); 1.6 (m, 1H); 1.7-1.85 (m, 3H); 1.9 (m, 1H); 2.0-2.1 (m, 1H); 2.2 (m, 1H); 2.5 (m, 1H); 2.6 (m, 2H); 2.7 (m, 1H), 3.1 (q, 1H); 3.4 (bs, 1H); 3.6 (m, 1H); 3.7 (t, 1H); 4.5 (t, 1H); 4.7 (bs, 1H); 7.1 (d, 2H); 7.2 (m, 1H); 7.35 (m, 2H) ppm. MS m/z 273 (M+1).

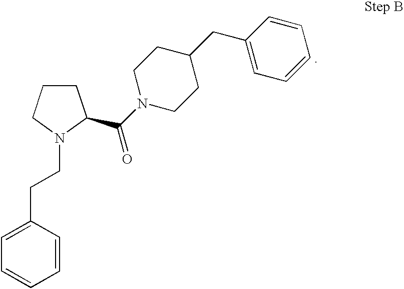

(4-Benzylpiperidin-1-yl)-((2S)-1-phenethylpyrrolidin-2-yl)-methanone (Compound 57).

We added 174 mg (0.56 mmol, 1.0 equivalent) (4-Benzyl-piperidin-1-yl)-(S)-pyrrolidin-2-yl-methanone, 0.085 mL (0.62 mmol, 1.1 equivalents) 2-bromoethylbenzene, and 270 mg (1.96 mmol, 3.5 equivalents) potassium carbonate to 10 mL acetonitrile. The solution was refluxed for 12 hours, filtered, and evaporated. The residue was dissolved in DCM, washed with saturated sodium bicarbonate, and the aqueous layer was extracted with DCM. We washed the combined organic phases with water and brine and then dried the organic phase over sodium sulfate. The solution was then filtered, and evaporated. The residue was purified via flash chromatography using a gradient from DCM to 4%MeOH in DCM. The fractions were evaporated, suspended in 5 mL Et₂O and dissolved by the dropwise addition of HCl/Et₂O. The ether was evaporated, the solid residue stirred in 10 mL diethyl ether for 30 min, decanted, and the ether wash was repeated. The solid was filtered and dried under reduced pressure to afford 96 mg (42%) of compound 57 as the HCl salt. ¹H NMR (CDCl₃, 500 MHz): [symbol] 1.0 (m, 2H); 1.7 (m, 3H); 1.9 (q, 1H); 2.1 (m, 1H); 2.2 (m, 1H); 2.3 (t, 0.5H); 2.5 (t, 2.5H); 2.6 (m, 1H); 2.7 (t, 0.5H); 2.9 (t, 0.5H); 3.1 (m, 1H); 3.2 (m, 1H); 3.3-3.8 (m, 5H); 4.4 (t, 1H); 4.6 (dd, 1H); 7.0 (d, 2H); 7.1-7.35 (m, 8H) ppm. MS m/z 377 (M+1).

### EXAMPLE 37

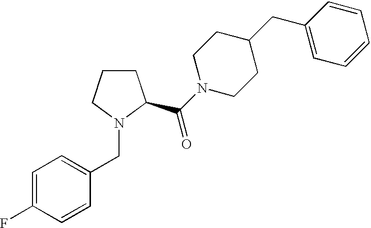

(4-Benzylpiperidin-1-yl)-[(2S)-1-(4-fluorobenzyl)-pyrrolidin-2-yl]-methanone Hydrochloride (Compound 58).

Compound 58 was prepared as described above except without heating and employing 4-flouro-benzyl-bromide instead of 2-bromoethyl-benzene, yielding 146 mg (70%) as the HCl salt. ¹H NMR (CDCl₃, 500 MHz): [symbol] 0.5-0.8 (m, 1.33H); 1.1 (m, 0.67H); 1.6-1.8 (m, 2H); 1.9 (m, 1H); 2.0 (m, 1H); 2.1 (m, 1H); 2.3 (m, 1H); 2.6 (m, 4H); 3.0 (q, 1H); 3.4 (m, 1H); 3.7 (dd, 1H); 3.8-4.1 (m, 2H); 4.3 (dd, 1H); 4.6 & 4.8 (dd, 1H); 4.7 (t, 1H); 7.2-7.4 (m, 5H); 7.45 (m, 2H); 7.6 (m, 2H) ppm. MS m/z 381 (M+1).

### EXAMPLE 38

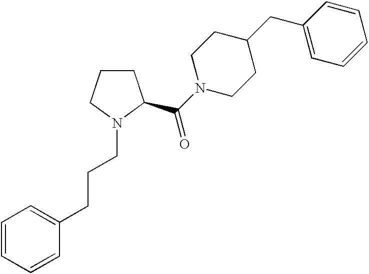

(4-Benzylpiperidin-1-yl)-[(2S)-1-(3-phenylpropyl)-pyrrolidin-2-yl]-methanone Hydrochloride (Compound 59).

Compound 59 was prepared as in Example 37, above, except heating only at 60° C. for 12 hours, and employing 3-phenylpropyl bromide instead of 2-bromoethylbenzene, yielding 190 mg (89%) as the HCl salt.

¹H NMR (CDCl₃, 500 MHz): □ 1.0 (m, 2H); 1.7 (m, 2H); 1.9-2.3 (m, 5H); 2.4-2.2.7 (m, 6H); 2.9 (m, 1H); 3.1-3.25 (m, 2H); 3.3 (m, 1H); 3.6 (bs, 1H); 3.7 (bs, 1H); 4.4 (d, 1H); 4.6 (bs, 1H); 7.0-7.3 (m, 10H) ppm. MS m/z 391 (M+1).

### EXAMPLE 39

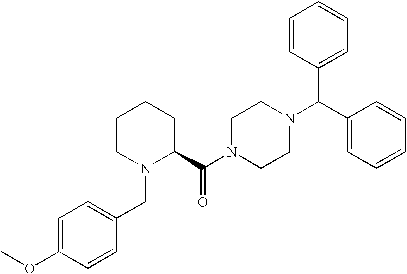

(4-Benzhydrylpiperazin-1-yl)-[(2S)-1-(4-methoxybenzyl)-piperidin-2-yl]-methanone Dihydrochloride (Compound 60).

Compound 60 was prepared from [4-(1,1-diphenylmethyl]-piperazin-1-yl]-(2S)-piperidin-2-yl-methanone and 4-methoxybenzyl bromide as described for Compound 21 in Example 9 to afford 141 mg (55%) as the dihydrochloride salt. ¹H NMR (DMSO-d₆, 500 MHz) δ 8.2 (m, 4H), 7.71 (m, 6H), 7.63 (dd, 2H), 7.28 (dd, 2H), 5.95 (m, 1H), 4.95-4.32 (m, 3H), 4.28 (m, 2H), 4.12 (m, 3H), 4.03 (s, 3H), 3.86 (m, 1H), 3.6-3.1 (m, 4H), 2.33 (m, 1H), 1.96 (m, 3H), 1.85 (m, 1H), 1.76 (m, 1H) ppm. MS m/z 484.5 (M+1)

### EXAMPLE 40

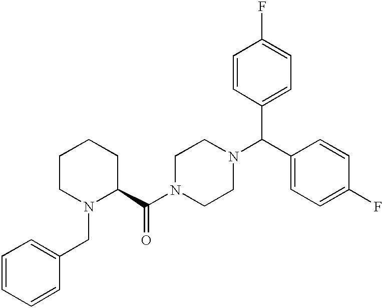

((2S)-1-Benzylpiperidin-2-yl)-{4-[bis-(4-fluorophenyl) methyl]-piperazin-1-yl}-methanone (Compound 61).

Compound 61 was prepared from {4-[Bis-(4-fluoro-phenyl)-methyl]-piperazin-1-yl}-(2S)-piperidin-2-yl-methanone and benzyl bromide as described for Compound 21 in Example 9 to afford 448 mg (75%) as the dihydrochloride salt. ¹H NMR (CDCl₃, 500 MHz) δ 7.16 (m, 9H), 6.81 (m, 4H), 4.02 (s, 1H), 3.68 (m, 1H), 3.46 (m, 2H), 3.00 (m, 1H), 2.73 (m, 1H), 2.14 (m, 4H), 1.8-1.04 (m, 6H) ppm. MS m/z 490.5 (M+1).

### EXAMPLE 41

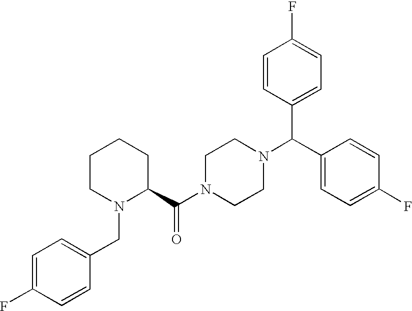

{4-[Bis-(4-fluorophenyl)methyl]-piperazin-1-yl}-[(2S)-1-(4-fluorobenzyl)-piperidin-2-yl]-methanone (Compound 62).

Compound 62 was prepared from {4-[Bis-(4-fluoro-phenyl)-methyl]-piperazin-1-yl}-(2S)-piperidin-2-yl-methanone and 4-fluorobenzyl bromide as described for Compound 21 in Example 9 to afford 510 mg (83%) as the dihydrochloride salt. ¹H NMR (CDCl₃, 500 MHz) δ 7.24 (m, 6H), 6.90 (m, 6H), 4.09 (s, 1H), 3.71 (m, 1H), 3.54 (m, 2H), 3.11 (m, 1H), 2.80 (m, 1H), 2.19 (m, 4H), 1.80-1.06 (m, 10H) ppm. MS m/z 508.5 (M+1).

### EXAMPLE 42

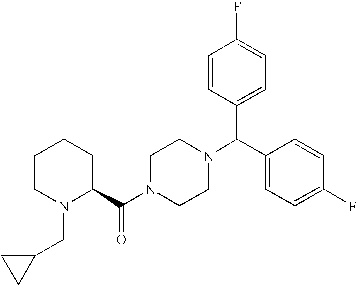

{4-[Bis-(4-fluorophenyl)methyl]-piperazin-1-yl}-((2S)-1-cyclopropylmethyl-piperidin-2-yl)-methanone (Compound 63).

Compound 63 was prepared from {4-[Bis-(4-fluoro-phenyl)-methyl]-piperazin-1-yl}-(2S)-piperidin-2-yl-methanone and cyclopropylmethyl bromide as described for Compound 21 in Example 9 to afford 442 mg (79%) as the dihydrochloride salt. ¹H NMR (CDCl₃, 500 MHz) δ 7.28 (m, 4H), 6.90 (m, 4H), 4.12 (s, 1H), 3.65 (m, 1H), 3.51 (m, 2H), 3.32 (m, 1H), 2.68 (m, 1H), 2.24 (m, 4H), 1.75-1.05 (m, 10H), 0.84 (m, 1H), 0.44 (m, 2H), 0.02 (m, 2H) ppm.

### EXAMPLE 43

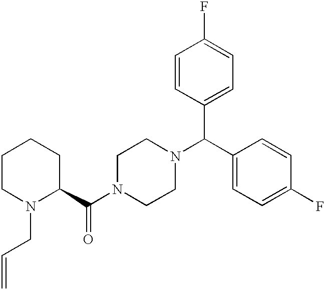

((2S)-1-Allylpiperidin-2-yl)-{4-[bis-(4-fluorophenyl)methyl]-piperazin-1-yl}-methanone (Compound 64).

Compound 64 was prepared from {4-[Bis-(4-fluorophenyl)-methyl]-piperazin-1-yl}-(2S)-piperidin-2-yl-methanone and allyl bromide as described for Compound 21 in Example 9 to afford 355 mg (65%) as the dihydrochloride salt. ¹H NMR (CDCl₃, 500 MHz) δ 7.31 (m, 4H), 6.96 (m, 4H), 5.81 (m, 1H), 5.09 (d, 2H), 4.17 (s, 1H), 3.87 (m, 1H), 3.66 (m, 1H), 3.57 (m, 1H), 3.50 (m, 1H), 3.22 (m, 1H), 3.06 (m, 1H), 2.78 (m, 1H), 2.26 (m, 4H), 1.95 (m, 1H), 1.84-1.34 (m, 6H), 1.22 (m, 1H) ppm. MS m/z 440.5 (M+1).

### EXAMPLE 44

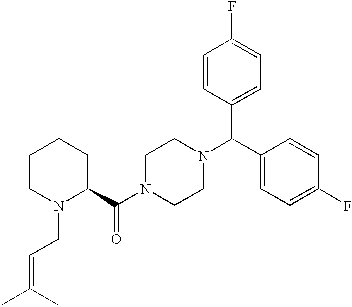

{4-[Bis-(4-fluorophenyl)methyl]-piperazin-1-yl}-[(2S)-1-(3-methyl-but-2-enyl)-piperidin-2-yl]-methanone (Compound 65).

Compound 65 was prepared from {4-[Bis-(4-fluoro-phenyl)-methyl]-piperazin-1-yl}-(2S)-piperidin-2-yl-methanone and 3-methyl-2-butenyl bromide as described for Compound 21 in Example 9 to afford 290 mg (51%) as the dihydrochloride salt. ¹H NMR (CDCl₃, 500 MHz) δ 7.50 (m, 4H), 7.13 (m, 4H), 5.38 (m, 1H), 4.34 (s, 1H), 3.88-3.60 (m, 3H), 3.54-2.98 (m, 3H), 2.46 (m, 4H), 1.95-1.00 (m, 9H), 1.85 (s, 3H), 1.70 (s, 3H) ppm. MS m/z 468.5 (M+1).

### EXAMPLE 45

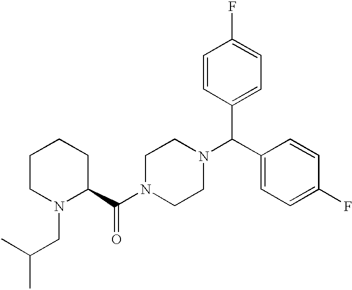

[4-[Bis-(4-fluorophenyl)methyl]-piperazin-1-yl]-((2S)-1-(2-methylpropyl)-piperidin-2-yl)-methanone (Compound 66).

Compound 66 was prepared similarly to Compound 21 (Example 9) from {4-[Bis-(4-fluoro-phenyl)-methyl]-piperazin-1-yl}-piperidin-2-yl-methanone (500 mg, 1.06 mmol) and l-bromo-2-methylpropane (164 mg, 1.22 mmol) to afford 590 mg (46% yield) after chromatography. ¹HNMR (CDCl₃, 500 MHz) δ 7.38-7.31, 4H m, 7.05-6.95, 4H m, 4.25-3.80, 2H m, 3.50-3.25 4H m, 3.20-2.75, 2H m, 2.42-2.25, 3H m, 2.25-1.70, 3H m, 1.62-1.40, 6H m, 1.38-1.00, 7H m ppm. MS: m/z 456.5 (M+1).

### EXAMPLE 46

Preparation of a Key Intermediate for the Compounds Synthesized By Scheme 5

The compounds described in Examples 46-59 were prepared by Scheme 5.

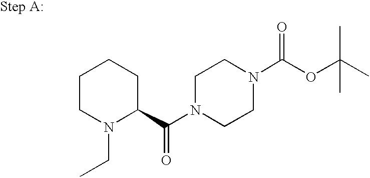

4-((2S)-1-Ethylpiperidine-2-carbonyl)-piperazine-1-carboxylic acid tert-butyl ester (Compound 67).

(2S)-1-Ethyl-piperidine-2-carboxylic acid (2.54 g, 16.24 mmol) was taken into 20 ml of dichloromethane and 10.4 ml (30 mmol) of diisopropylethylamine. Pivaloyl chloride (2 ml, 16.24 mmol) was added to the solution dropwise. After stirring at room temperature for 1 hour, a solution of piperazine-1-carboxylic acid tert-butyl ester (2.76 g, 14.6 mmol) was added dropwise and the reaction was stirred overnight. The reaction was washed with 1N sodium hydroxide, water, and brine. The organic layer was dried over anhydrous sodium sulfate, filtered and evaporated in vacuo to afford a yellow oil which was purified by flash chromatography (SiO₂) eluting with gradient of dichloromethane to 5% methanol to afford 4.7 g (98%) of compound 67. ¹H NMR (CDCl₃, 500 MHz) δ 4.08 (m, 1H), 3.83 (m, 1H), 3.64 (m, 1H),3.56-3.40 (m, 6H), 3.13 (m, 2H), 2.68 (m, 1H), 2.24 (m, 1H), 1.94 (m, 1H), 1.80 (m, 2H), 1.66 (m, 2H), 1.48 (s, 9H), 1.32 (m, 1H), 1.09 (m, 3H) ppm.

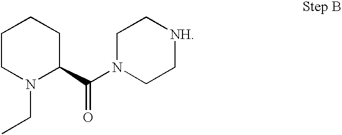

((2S)-1-Ethylpiperidin-2-yl)-piperazin-1-yl-methanone dihydrochloride (Compound 68).

4-((2S)-1-Ethyl-piperidine-2-carbonyl)-piperazine-1-carboxylic acid tert-butyl ester (3.1 g, 9.5 mmol) was dissolved in 50 mL EtOAc and treated with HCl (g). After stirring for 1 hour, the resulting precipitate was filtered, washed with EtOAc, and dried in vacuo yielding 1.19 g (55%) of compound 68. MS: m/z (M+1) 299.

### EXAMPLE 47

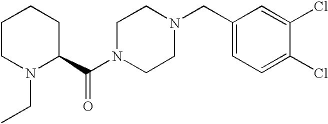

[4-(3,4-Dichlorobenzyl)-piperazin-1-yl]-((2S)-1-ethylpiperidin-2-yl)-methanone Dihydrochloride (Compound 69).

((S)-1-Ethyl-piperidin-2-yl)-piperazin-1-yl-methanone dihydrochloride (200 mg (0.70 mmol, 1 equivalent), 139 mg (0.70 mmol, 1.0 equivalent) 3,4-dichlorobenzyl chloride, and 340 mg (2.5 mmol, 3 equivalents) potassium carbonate were suspended in 10 mL acetonitrile and stirred at 60° C. for 5 hours. The reaction was filtered through Celite and evaporated in vacuo to afford an oil that was dissolved in DCM, washed with saturated sodium bicarbonate, and brine. The combined organic phases were washed with water, then brine. The washed organic phase was then dried over sodium sulfate, filtered, and evaporated. The resulting crude residue was purified via flash chromatography using a gradient from DCM to 6% MeOH in DCM. The product was then dissolved in Et₂O and HCl/Et₂O was added drop-wise until no more precipitate formed. The precipitate was removed by filtration and the filtrate was lyophilized to yield 35 mg (11%) of compound 69 as the dihydrochloride salt. ¹H NMR (CDCl₃, 500 MHz): [symbol] 1.3 (t, 3H); 1.6 (t, 1H); 1.8 (m, 2H); 2.0 (dd, 2H); 2.2 (dd, 2H); 3.1 (m, 2H); 3.2 (m, 1H); 3.5 (bs, 4.5H); 3.8 (d, 1H); 3.9 (bs, 3.5H); 4.4 (s, 2H); 4.5 (d, 1H); 7.5 (d, 1H); 7.70 (d, 1H); 7.75 (s, 1H) ppm. MS m/z 386 (M+1).

### EXAMPLE 48

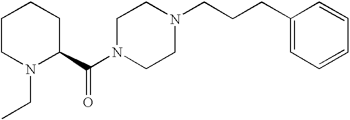

((2S)-1-Ethylpiperidin-2-yl)-[4-(3-phenylpropyl)-piperazin-1-yl]-methanone dihydrochloride (Compound 70).

Compound 70 was prepared as described in Example 47 employing (3-bromo-propyl)-benzene instead of 3,4-dichloro-benzyl chloride to yield 102 mg (37%) as the dihydrochloride salt. ¹H NMR (CDCl₃, 500 MHz): [symbol] 1.3 (t, 3H); 1.6 (t, 1H); 1.8 (m, 2H); 2.0 (dd, 2H); 2.1 (m, 3H); 2.7 (t, 2H); 2.8-3.3 (m, 8H); 3.7 (m, 4H); 4.2 (bs, 1H); 4.4 (d, 1H); 4.6 (bs, 1H); 7.3 (m, 3H); 7.4 (m, 2H). MS m/z 417 (M+1).

### EXAMPLE 49

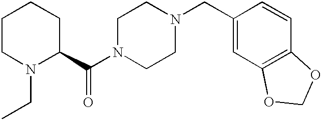

(4-Benzo[1,3]dioxol-5-ylmethylpiperazin-1-yl)-((2S)-1-ethylpiperidin-2-yl)-methanone Dihydrochloride (Compound 71).

Compound 71 was prepared as described in Example 47 employing 5-chloromethyl-benzo[1,3]dioxole instead of 3,4-dichloro-benzyl chloride to yield 196 mg (68%) as the dihydrochloride salt. ¹H NMR (CDCl₃, 500 MHz): □ 1.4 (t, 3H); 1.7 (t, 1H); 1.9 (m, 2H); 2.1 (dd, 2H); 2.3 (d, 1H); 3.1 (m, 2.5H); 3.3 (m, 1.5H); 3.3-3.8 (m, 4H); 3.85 (d, 1.5H); 3.9-4.3 (m, 1.5H); 4.4 (s, 2H); 4.6 (m, 2H); 6.1-6.3 (3 s, 2H); 7.0-7.3 (m, 3H) ppm. MS m/z 360 (M+1).

### EXAMPLE 50

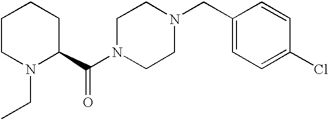

[4-(4-Chlorobenzyl)-piperazin-1-yl]-((2S)-1-ethylpiperidin-2-yl)-methanone Dihydrochloride (Compound 72).

Compound 72 was prepared as described in Example 47 employing 4-chloro-benzyl-bromide instead of 3,4-dichloro-benzyl chloride to yield 44 mg (16%) as the dihydrochloride salt. ¹H NMR (CDCl₃, 500 MHz): □ 1.3 (t, 3H); 1.6 (t, 1H); 1.8 (m, 2H); 2.0 (dd, 2H); 2.2 (dd, 2H); 3.1 (m, 2H); 3.2 (m, 1H); 3.5 (bs, 4.5H); 3.8 (d, 1H); 3.9 (bs, 3.5H); 4.4 (s, 2H); 4.5 (d, 1H); 7.4 (d, 2H); 7.5 (d, 2H) ppm. MS m/z 423 (M+1).

### EXAMPLE 51

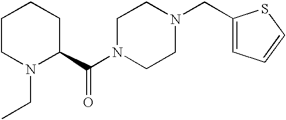

((2S)-1-Ethylpiperidin-2-yl)-(4-thiophen-2-ylmethylpiperazin-1-yl)-methanone Dihydrochloride (Compound 73).

Compound 73 was prepared as described in Example 47 employing 2-chloromethyl-thiophene instead of 3,4-dichloro-benzyl chloride. 2-chloromethyl-thiophene was prepared as described in J. Janusz et al., J. Med. Chem., 41, pp. 3515-3529 (1998). This process yielded 93 mg (50%) of compound 73. ¹H NMR (CDCl₃, 500 MHz): [symbol] 1.2 (t, 3H); 1.5 (t, 1H); 1.6 (q, 2H);1.8 (dd, 2H); 2.0 (d, 1H); 2.9 (m, 2H), 3.1 (bs, 4H); 3.4-3.7 (m, 4H); 4.1 (bs, 1H), 4.3 (d, 1H) 4.5 (s, 4H); 7.0 (dd, 1H); 7.2 (dd, 1H); 7.5 (d, 1H) ppm. MS m/z 317 (M+1).

### EXAMPLE 52

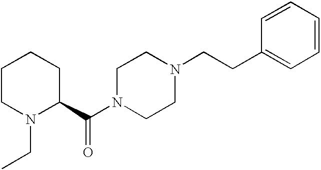

((2S)-1-Ethylpiperidin-2-yl)-(4-phenethylpiperazin-1-yl)-methanone Dihydrochloride (Compound 74).

Compound 74 was prepared as described in Example 47 employing phenethyl bromide instead of 3,4-dichlorobenzyl chloride to yield 158 mg (50%). ¹H NMR (DMSO-d₆) δ 12.2 (br s, 1H), 9.7 (br s, 1H), 7.51 (m, 2H), 7.41 (m, 3H), 4.95 (m, 0.5H), 4.76 (m, 0.5H), 4.61 (m, 1H), 4.32 (m, 1H), 4.22 (m, 2H), 3.86 (m, 1H), 3.80 (m, 1H), 3.71 (m, 1H), 3.47 (m, 3H), 3.31-2.98 (m, 6H), 2.22 (m, 1H), 1.93 (m, 3H), 1.72 (m, 2H), 1.38 (t, 3H) ppm. MS m/z 330.5 (M+1).

### EXAMPLE 53

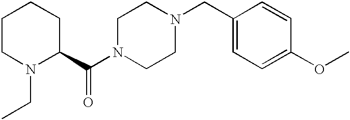

((2S)-1-Ethylpiperidin-2-yl)-[4-(4-methoxybenzyl)-piperazin-1-yl]-methanone (Compound 75).

Compound 75 was prepared as described in Example 47 employing 4-methoxybenzyl chloride instead of 3,4-dichlorobenzyl chloride to yield 133 mg (47%). ¹H NMR (CDCl₃, 500 MHz) δ 7.16 (d, 2H), 6.8 (d, 2H), 3.91 (m, 1H), 3.76 (s, 3H), 3.58 (m, 1H), 3.53 (m, 1H), 3.41 (s, 2H), 3.06 (m, 2H), 2.58 (m, 1H), 2.33 (m, 4H), 2.14 (m, 1H), 1.9-1.4 (m, 6H), 11.2 (m, 2H), 0.98 (m, 3H) ppm. MS m/z 346.4 (M+1).

### EXAMPLE 54

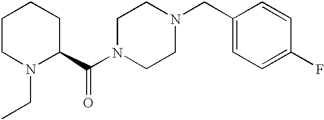

((2S)-1-Ethylpiperidin-2-yl)-[4-(4-fluorobenzyl)-piperazin-1-yl]methanone (Compound 76).

Compound 76 was prepared as described in Example 47 using 4-fluorobenzyl bromide instead of 3,4-dichloro-benzyl chloride to yield 134 mg (49%). ¹H NMR (CDCl₃, 500 MHz) δ 7.2 (m, 2H), 6.96 (m, 2H), 3.92 (m, 1H), 3.73 (m, 1H) 3.59 (m, 1H), 3.53 (m, 1H), 3.41 (s, 2H), 3.05 (m, 2H), 2.58 (m, 1H), 2.28 (m, 4H), 2.15 (m, 1H), 1.82 (m, 1H), 1.75-1.38 (m, 5H), 1.22 (m, 1H), 0.95 (m, 3H) ppm. MS m/z 334.4 (M+1).

### EXAMPLE 55

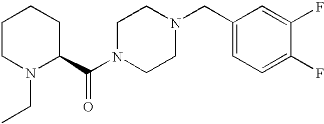

[4-(3,4-Difluorobenzyl)-piperazin-1-yl]-((2S)-1-ethylpiperidin-2-yl)-methanone (Compound 77).

Compound 77 was prepared as described in Example 47 using 3,4-difluorobenzyl bromide instead of 3,4-dichloro-benzyl chloride to yield 185 mg (65%).

¹H NMR (CDCl₃, 500 MHz) δ 7.28 (m, 1H), 7.18 (m, 1H), 7.10 (m, 1H), 4.06 (m, 1H), 3.88 (m, 1H), 3.75 (m, 1H), 3.66 (m, 1H), 3.52 (s, 2H), 3.18 (m, 2H), 2.72 (m, 1H), 2.45 (m, 4H), 2.25 (m, 1H), 1.94 (m, 1H), 1.88-1.55 (m, 5H), 1.34 (m, 1H), 1.08 (m, 3H) ppm. MS m/z 352.5 (M+1).

### EXAMPLE 56

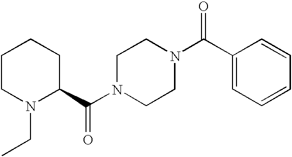

[4-((2S)-1-Ethylpiperidine-2-carbonyl)-piperazin-1-yl]-phenyl Methanone (Compound 78).

((2S)-1-Ethyl-piperidin-2-yl)-piperazin-1-yl-methanone dihydrochloride (221 mg, 0.74 mmol) was suspended in 5 mL anhydrous DCM. N,N-diisopropylethylamine (0.45 ml, 2.6 mmol) was added to the solution followed by the dropwise addition of benzoyl chloride (0.095 ml, 0.81 mmol). After stirring at room temperature for 16 hours, the reaction was diluted with 5 mL of dichloromethane and washed with saturated sodium bicarbonate, water, and brine. The organic layer was dried over anhydrous sodium sulfate, filtered, and evaporated in vacuo. The crude residue was purified by flash chromatography (SiO₂) using a gradient from 100% dichloromethane to 6% methanol in dichloromethane to afford 110 mg (45%) of the compound 78. ¹H NMR (CDCl₃, 500 MHz): [symbol] 1.0 (t, 3H); 1.1-1.9 (m, 7H); 2.1 (bs, 1H); 2.7 (bs, 1H); 3.0 (bs, 2H); 3.2-3.9 (m, 7H); 4.1 (bs, 1H); 7.4 (m, 5H) ppm. MS m/z 330 (M+1).

### EXAMPLE 57

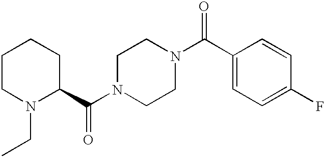

((2S)-1-Ethylpiperidin-2-yl)-[4-(4-fluorobenzoyl)-piperazin-1-yl]methanone Hydrochloride (Compound 79).

Compound 79 was prepared as described in Example 56 using 4-flourobenzoyl chloride instead of benzoyl chloride to yield 148 mg (54%) as the hydrochloride salt. ¹H NMR (CDCl₃, 500 MHz): □ 1.2 (t, 3H); 1.5 (m, 1H); 1.65 (broad t, 2H); 1.85 (m, 2H); 2.1 (m, 1H); 2.9 (m, 2H); 3.1 (m, 1H); 3.5 (m, 4H); 3.7 (m, 5H); 4.3 (m, 1H); 7.1 (t, 2H); 7.4 (m, 2H). %) ppm. MS m/z 348 (M+1).

### EXAMPLE 58

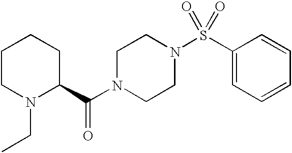

(4-Benzenesulfonylpiperazin-1-yl)-((2S)-1-ethylpiperidin-2-yl)-methanone hydrochloride (Compound 80).

Compound 80 was prepared as described in Example 56 using benzenesulfonyl chloride instead of 4-flourobenzoyl chloride to yield 117 mg (45%) as the HCl salt. ¹H NMR (CDCl₃, 500 MHz): □ 0.85 (t, 3H); 1.1-1.2 (m, 1.5H); 1.4-1.55 (m, 2.5H); 1.6 (d, 1H); 1.7 (d, 1H); 1.8 (t, 1H); 2.0 (m, 1H); 2.4 (m, 1H); 2.9 (bs, 2H); 3.0 (d, 4H); 3.5-3.8 (broad dd, 2H); 3.9 (bs, 1H); 4.1 (bs, 1H); 7.5 (t, 2H); 7.6 (t, 1H); 7.7 (d, 2H) ppm. MS m/z 366 (M+1).

### EXAMPLE 59

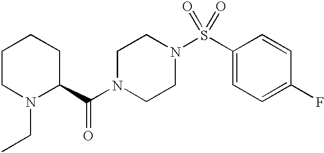

((2S)-1-Ethylpiperidin-2-yl)-[4-(4-fluorobenzenesulfonyl)-piperazin-1-yl]-methanone Hydrochloride (Compound 81).

Compound 81 was prepared as described in Example 56 using 4-flourobenzenesulfonyl chloride instead of 4-flourobenzoyl chloride to yield 181 mg (67%) as the HCl salt. ¹H NMR (CDCl₃, 500 MHz): [symbol] 1.0 (t, 3H); 1.2-1.5 (m, 3H); 1.6 (d, 1H); 1.7-1.8 (m, 2H); 2.7 (m, 2H); 2.85 (m, 3H); 2.95 (m, 2H); 3.4-3.6 (m, 5H); 4.1 (m, 1H); 7.2 (t, 2H); 7.7 (m, 2H) ppm. MS m/z 384 (M+1).

The compounds described in Examples 60-64 were prepared using the synthetic scheme depicted in Scheme 6.

### EXAMPLE 60

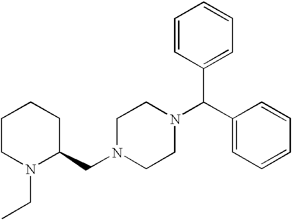

1-Benzhydryl-4-((2S)-1-ethylpiperidin-2-ylmethyl)-piperazine (Compound 100).

10 ml (10 mmol) of 1M Borane-tetrahydrofuran complex was added to a solution of 150 mg (0.36 mmol) of 1-[4-(1,1-Diphenylmethyl)piperazin-1-yl]-1-((S)-1-ethylpiperidin-2-yl)methanone (Compound 1) in 10 ml of anhydrous THF at room temperature. The reaction was stirred for 4 days then quenched with the dropwise addition of methanol. The mixture was evaporated in vacuo to give a clear viscous oil. The crude product was dissolved in 10 ml of 1N HCl and 1 ml of acetone was added and the solution stirred for 30 mins. The mixture was basified with saturated sodium bicarbonate and then extracted with dichloromethane (2×). The combined extracts were washed with brine, dried over anhydrous sodium sulfate, filtered and evaporated to afford a clear oil that was purified by flash chromatography(SiO₂) eluting with 100:5 dichloromethane/methanol to afford 72 mg of the title compound.

¹H NMR (CDCl₃, 500 MHz) δ 7.31 (m, 4H), 7.18 (m, 4H), 7.11 (m, 2H), 4.10 (s, 1H), 3.15-2.60 (m, 5H), 2.58-2.08 (m, 10H), 1.8 (m, 2H), 1.72 (m, 3H), 1.29 (m, 1H), 1.13 (m, 3H) ppm. MS: m/z (M+1) 378.5

### EXAMPLE 61

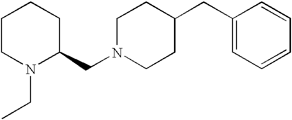

4-Benzyl-1-((2S)-1-ethylpiperidin-2-ylmethyl)-piperidine (Compound 101).

Compound 101 was prepared by the reduction of compound 26 as described in Example 60 to yield 141 mg.

¹H NMR (DMSO-d₆, 500 MHz) δ 7.45 (m, 2H), 7.36 (m, 3H), 4.23 (m, 3H), 3.99 (m, 1H), 3.88-3.68 (m, 2H), 3.64 (m, 1H), 3.53-3.22 (m, 2H), 3.10 (m, 2H), 2.64 (m, 3H), 2.44 (m, 0.5H), 2.22 (m, 0.5H), 2.07-1.61 (m, 9H),1.43 (t, 3H) ppm. MS m/z (M+1) 301.5

### EXAMPLE 62

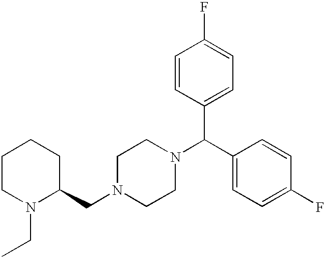

1-[Bis-(4-fluorophenyl)methyl]-4-((2S)-1-ethylpiperidin-2-ylmethyl)-piperazine. (Compound 102).

Compound 102 was prepared by the reduction of Compound 25 as described in Example 60 to yield 369 mg.

¹H NMR (CD₃OD, 500 MHz) δ 7.72 (m, 4H), 7.12 (m, 4H), 5.48 (d, 1H), 3.63 (br s, 0.5H), 3.43 (m, 1H), 3.34 (m, 1.5H), 3.22-2.75 (m, 11H), 2.62 (m, 1H), 1.95 (m, 0.5H), 1.86 (m, 0.5H), 1.72-1.58 (m, 3H), 1.48 (m, 2H), 1.25 (m, 3H) ppm. MS: m/z (M+1) 414.6

### EXAMPLE 63

Synthesis of ((2S,4R)-1-Benzyl-4-methoxypyrrolidin-2-yl)-(4-benzylpiperidin-1-yl)methanone (Compound 153)

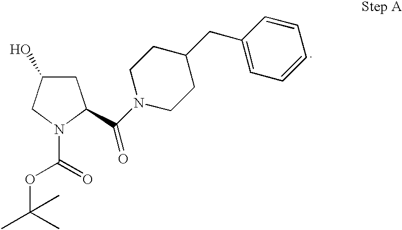

(2S,4R)-2-(4-benzylpiperdine-1-carbonyl)-4-hydroxypyrrolidine-1-carboxylic Acid Tert-Butyl Ester (Compound 151).

To Boc-4-hydroxyproline (5.0 g, 21.6 mmol) in 20 mL of dichloromethane was added diisopropyl carbodiimide (3.0 g, 23.9 mmol) and 1-Hydroxylbenzotriazole (3.2 g, 23.8 mmol). After stirring for 1 h, 4-benzylpiperdine (4.2 g, 23.8 mmol) was added neat. The solution was stirred for 12 hours. The reaction was diluted with 50 ml of dichloromethane and washed with 1M HCl, NaHCO₃ (sat.), brine, dried (MgSO₄) and concentrated. The product was purified by flash chromatography to give 6.67 g (80% yield) as a white foam. ¹H NMR (500 MHz, CDCl₃) δ 7.45-7.20 (m, 5H), 5.40 (s, 1H), 4.90-4.45 (m, 2H), 4.10-3.55 (m, 3H), 3.30-3.00 (m, 1H), 2.75-2.55 (m, 2H), 2.35-1.70 (m, 7H), 1.60&1.50 (s, s 9H (rotomers)), 1.40-1.10 (m, 2H) ppm. MS: m/z 389.5 (M+1).

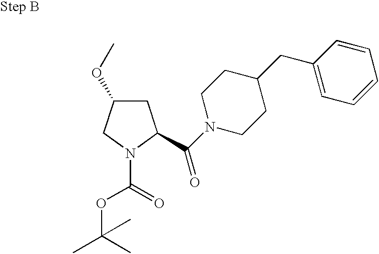

(2S,4R)-2-(4-benzylpiperdine-1-carbonyl)-4-methoxypyrrolidine-1-carboxylic Acid Tert-Butyl Ester (Compound 152).

2-(4-benzylpiperdine-1-carbonyl)-4-hydroxy-pyrrolidine-1-carboxylic acid tert-butyl ester in THF (5 mL) was added dropwise to hexane-washed NaH (113 mg, 2.83 mmol) suspended in THF (5 mL). After stirring for 0.5 h, methyl iodide (402 mg, 2.83 mmol) was added neat and the solution was refluxed for 4 hours. The reaction was poured into NaHCO₃ (sat.), extracted with ethyl acetate, washed with brine, dried (MgSO₄) and concentrated. Flash chromatography afforded 720 mg (70% yield) of a amber oil. ¹H NMR (500 MHz, CDCl₃) δ 7.20-6.90 (m, 5H), 4.90-4.45 (m, 2H), 4.15-3.50 (m, 3H), 3.35&3.31 (s, s, 3H (rotomers)), 3.20-2.90 (m, 1H), 2.60-2.50 (m, 2H), 2.30-1.65 (m, 6H), 1.60&1.50 (s, s 9H (rotomers)), 1.40-1.10 (m, 2H) ppm. MS: m/z 403.5 (M+1).

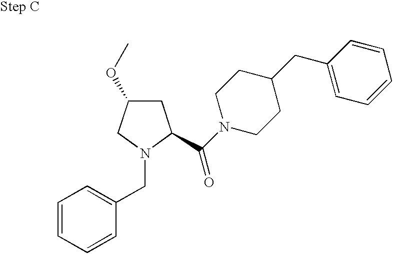

((2S,4R)-1-Benzyl-4-methoxypyrrolidin-2-yl)-(4-benzylpiperdin-1-yl)-methanone (Compound 153).

2-(4-benzylpiperdine-1-carbonyl)-4-methoxy-pyrrolidine-1-carboxylic acid tert-butyl ester (720 mg, 1.79 mmol) was treated with HCl(g) in ethyl acetate. After 1 hour the solution was evaporated and used without further purification. Alkylation was performed as described above from (4-Benzyl-piperidin-1-yl)-(4-methoxy-pyrrolidin-2-yl)-methanone and Benzyl bromide (459 mg, 2.68 mmol) to afford 400 mg after flash chromatography. The final compound 153 was converted to a citrate salt (592 mg, 57% yield) ¹HNMR (500 MHz, CDCl₃) δ 7.20-6.90 (m, 10H), 4.50-4.40 (d, 2H), 4.90-3.10 (m, 5H), 3.05 (s, 3H), 2.70-2.60 (m, 1H), 2.00-1.80 (m, 1H), 1.60-1.35 (m, 4H), 1.30-1.10 (m, 2H) ppm. MS: m/z 393.5 (M+1).

### EXAMPLE 64

Synthesis of [(2S, 4R)-1-Benzyl-5-(4-benzylpiperidine-1-carbonyl)-pyrrolidin-3-yloxyl]-acetic Acid Methyl Ester (Compound 155).

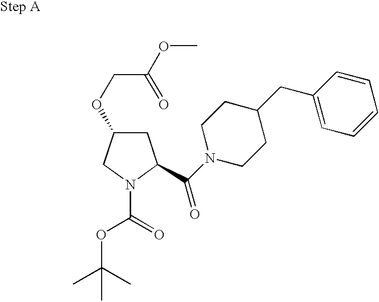

(2S, 4R)-2-(4-benzylpiperdine-1-carbonyl)-4-methoxycarbonylmethoxypyrrolidine-1-carboxylic Acid Tert-Butyl Ester (Compound 154).

This was prepared via the procedure reported for Example 63 (Step B) where the reaction of Compound 151 (1.0 g, 2.57 mmol) and methyl bromo acetate (488 mg, 5.14 mmol) afforded 581 mg (49% yield) of the desired product. ¹H NMR (500 MHz, CDCl₃) δ 7.40-7.30 (m, 5H), 4.95-4.65 (m, 2H), 4.45-3.65-(m, 8H), 3.20-2.95 (m, 2H), 2.70-2.20 (m, 5H), 1.90-1.70 (m, 3H), 1.60-1.45 (s, s, 9H(rotomers)), 1.45-1.15 (m, 2H) ppm. MS: m/z 461.5 (M+1).

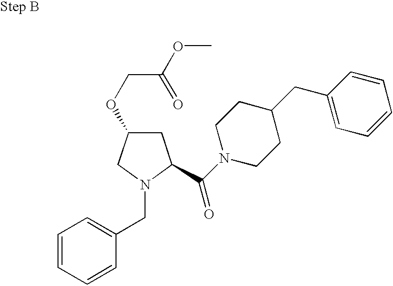

[(2S, 4R)-1-Benzyl-5-(4-benzylpiperidine-1-carbonyl)-pyrrolidin-3-yloxyl]-acetic Acid Methyl Ester (Compound 155).

Compound 155 was prepared as described in Example 63, Step C from 2-(4-benzyl-piperdine-1-carbonyl)-4-methoxy-carbonylmethoxy-pyrroline-1-carboxylic acid tert-butyl ester (581 mg, 1.26 mmol) and Benzyl bromide (324 mg, 1.89 mmol) to afford 270 mg (48% yield) after flash chromatography. ¹H NMR (500 MHz, CDCl₃) δ 7.40-6.95 (m, 10H), 4.50-4.40 (m, 1H), 4.10-3.65 (m, 5H), 3.70-3.20 (m, 5H), 2.71-2.62 (m, 1H), 2.40-2.35 (m, 3H), 2.20-1.90 (m, 2H), 1.60-1.40 (m, 4H), 1.20-1.00 (m, 3H) ppm. MS: m/z 451.5 (M+1).

### EXAMPLE 65

Combinatorial Synthesis of Compounds Via Scheme 7

Compounds of this invention were also made via the synthetic scheme set forth in Scheme 7. The coupling of the appropriate Boc-Amino Acid (150 □mol) with amines (300 □mole) was accomplished using N-cyclohexanecarbodiimide-N′-propyloxymethyl polystyrene resin (300 □mol) as described in Example 12. The resulting Boc-protected amino amides were treated with a saturated solution of HCl in ethyl acetate (5 mL). After shaking for 3 hours, filtration and evaporation afforded the pure products as hydrogen chloride salts.

The above products were taken up in methanol (1 mL) and transferred to the reaction block wells containing K₂CO₃ (excess) suspended in CH₃CN (5 mL). The reactions were treated with the appropriate alkyl halide (300 □mol) and the reaction block was shaken for 24 hours at ambient temperature or at 50° C., depending upon the alkyl halide. Filtration and evaporation gave the crude compounds. Purification was performed using reverse phase HPLC (H₂O/CH₃CN/0.1% TFA) to afford the desired products as determined by LC/MS.

Table 3 sets forth compounds that were prepared by this method or via Scheme 3 (see, Example 11) and their mass spectrometry values.

TABLE 3
Compounds prepared by Scheme 3 (N-methyl derivatives) Scheme 7 (N-ethyl or N-benzyl derivatives).

| # | Structure | MS (m/z) | # | Structure | MS (m/z) |
|---|---|---|---|---|---|
| 200 | 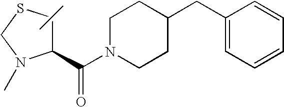 | 333.51 | 201 | 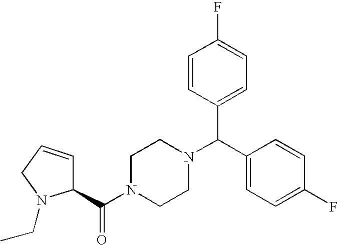 | 411.56 |
| 202 | 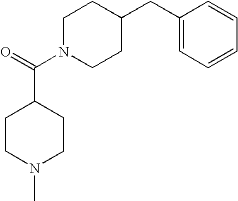 | 301.57 | 203 | 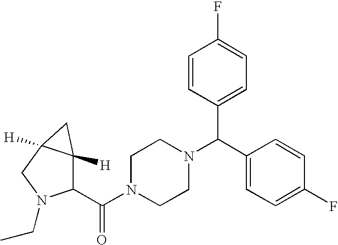 | 426.58 |
| 204 | 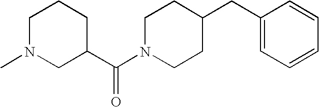 | 301.57 | 205 | 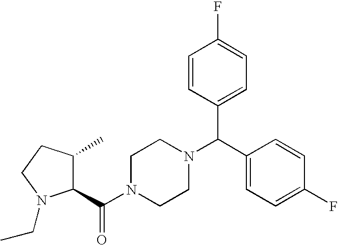 | 428.59 |
| 206 | 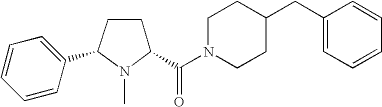 | 363.55 | 207 | 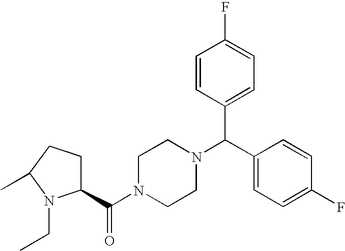 | 428.59 |
| 213 | 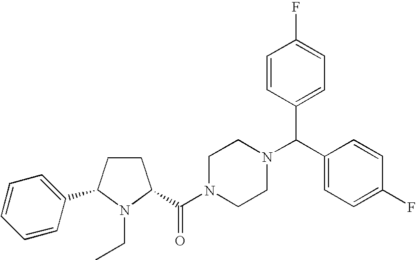 | 490.56 | 209 | 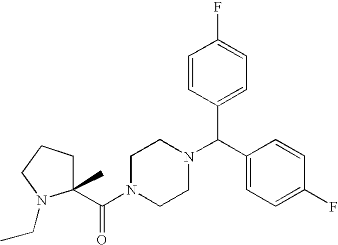 | 428.59 |
| 215 | 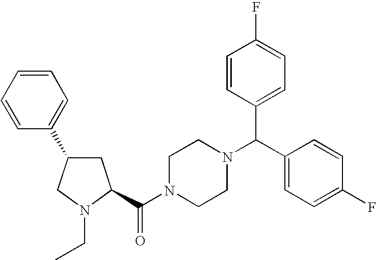 | 490.57 | 211 | 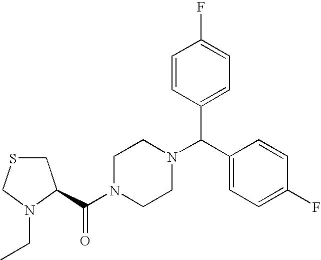 | 432.52 |
| 216 | 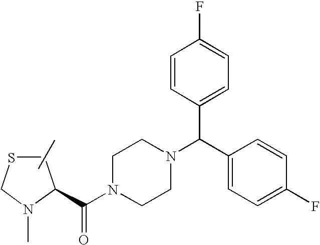 | 446.45 | 217 | 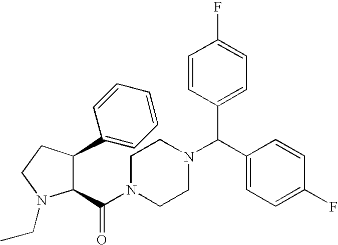 | 490.57 |
| 218 | 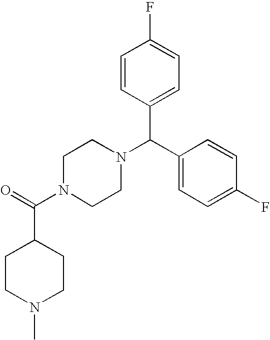 | 414.51 | 219 | 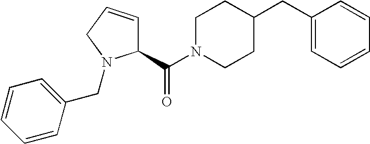 | 359.56 |
| 220 | 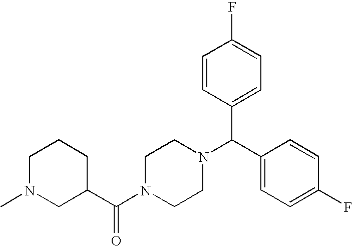 | 414.52 | 221 | 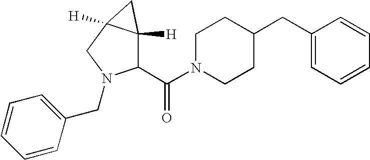 | 375.60 |
| 222 | 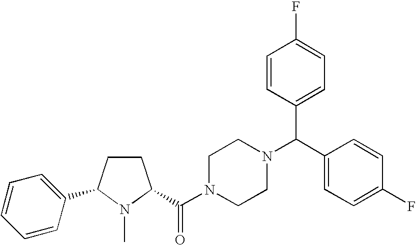 | 476.5 | 223 | 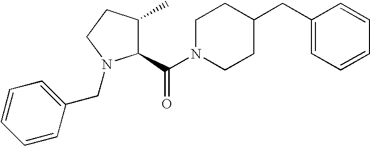 | 377.50 |
| 224 | 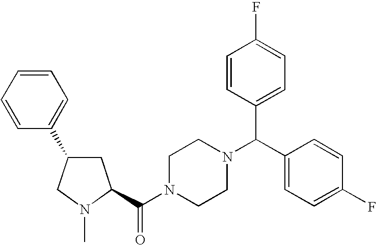 | 476.5 | 225 | 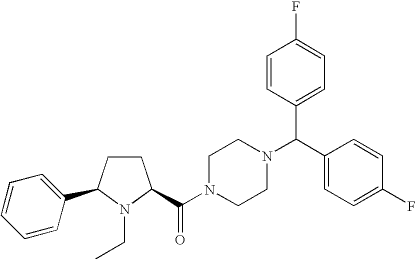 | 490.57 |
| 226 | 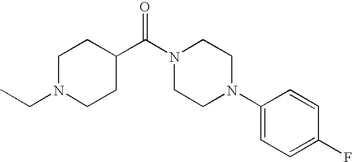 | 320.1 | 227 | 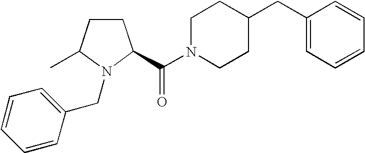 | 377.60 |
| 228 | 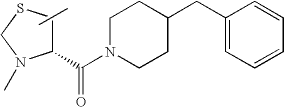 | 333.51 | 229 | 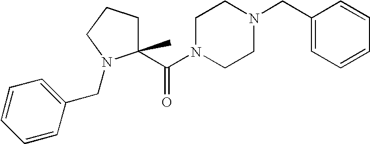 | 377.60 |
| 230 | 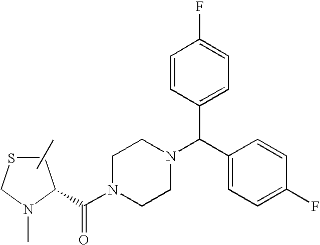 | 446.45 | 231 | 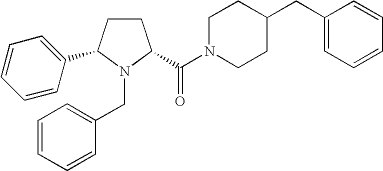 | 439.59 |
| 232 | 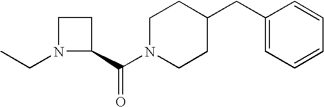 | 287.54 | 233 | 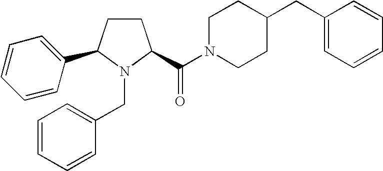 | 439.59 |
| 234 | 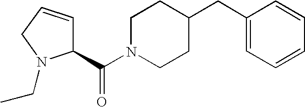 | 299.53 | 235 | 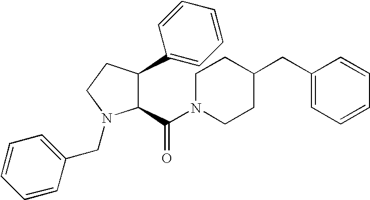 | 439.59 |
| 236 | 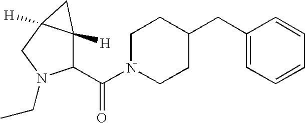 | 313.57 | 237 | 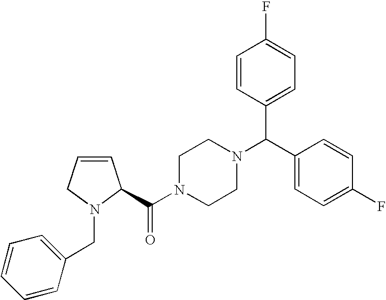 | 474.52 |
| 238 | 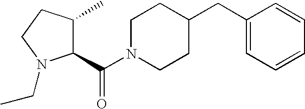 | 315.60 | 239 | 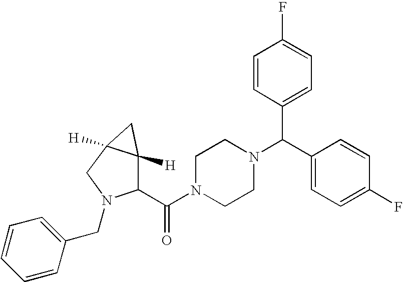 | 488.55 |
| 240 | 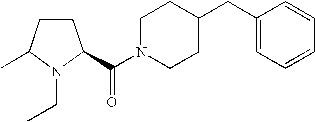 | 315.60 | 241 | 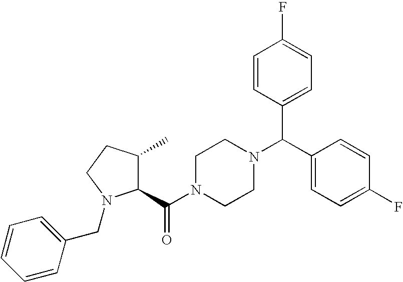 | 490.57 |
| 242 | 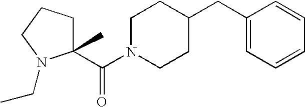 | 315.60 | 243 | 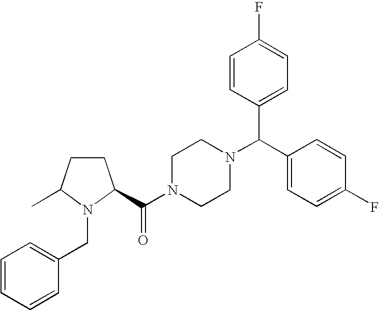 | 490.57 |
| 244 | 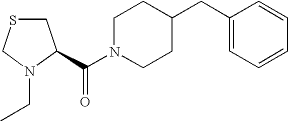 | 319.53 | 245 | 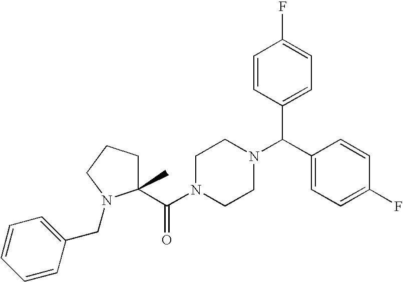 | 490.57 |
| 246 | 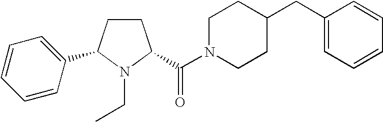 | 377.60 | 247 | 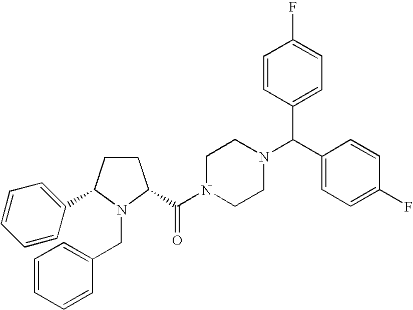 | 552.54 |
| 248 | 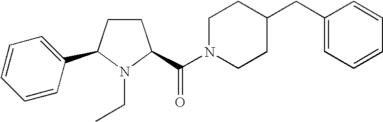 | 377.60 | 249 | 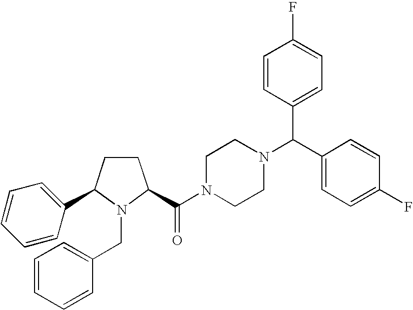 | 552.54 |
| 250 | 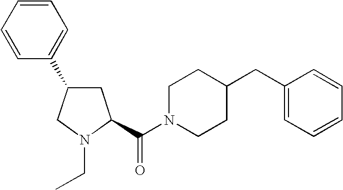 | 377.60 | 251 | 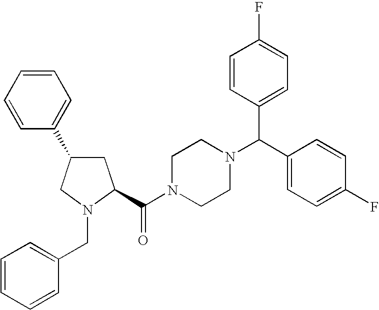 | 552.54 |
| 252 | 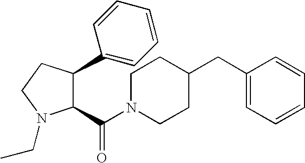 | 377.60 | 253 | 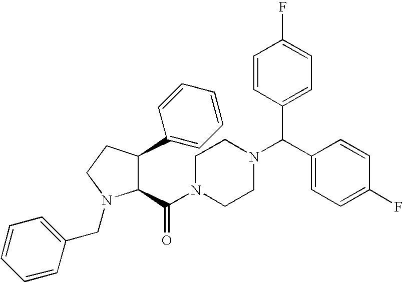 | 552.54 |
| 254 | 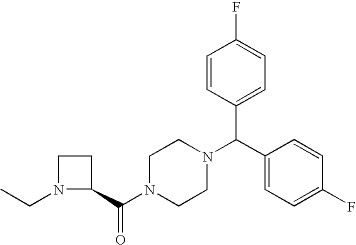 | 400.57 | 255 | 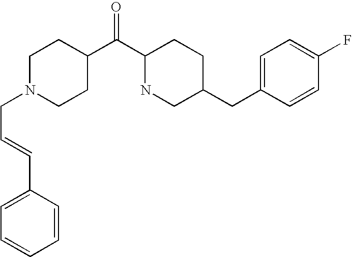 | 420.1 |
| 256 | 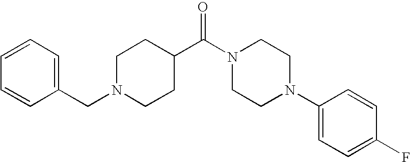 | 382.1 | 257 | 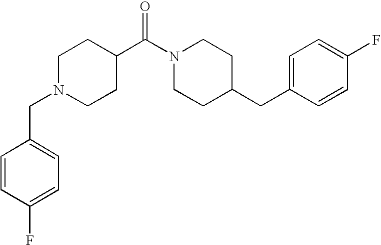 | 413.1 |
| 258 | 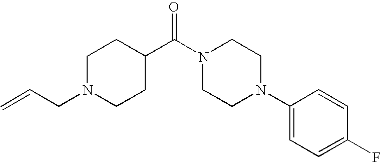 | 332.0 | 259 | 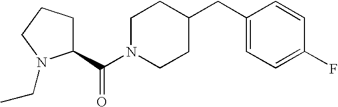 | 319.1 |
| 260 | 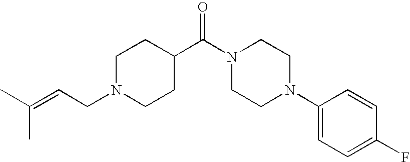 | 360.2 | 261 | 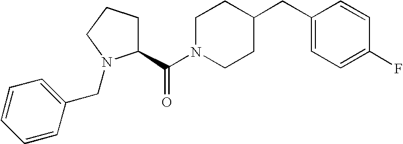 | 381.1 |
| 262 | 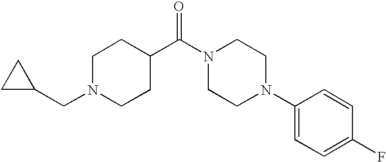 | 346.1 | 263 | 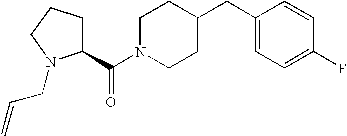 | 331.1 |
| 264 | 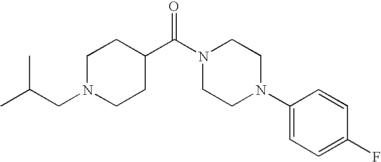 | 346.1 | 265 | 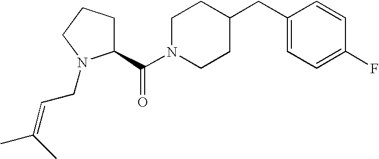 | 359.1 |
| 266 | 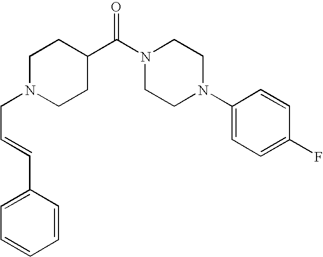 | 408.1 | 267 | 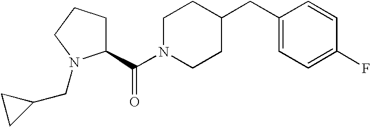 | 345.1 |
| 268 | 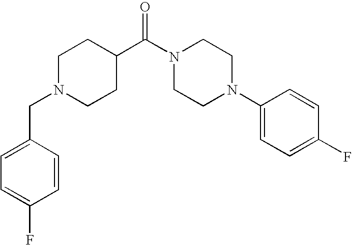 | 410.1 | 269 | 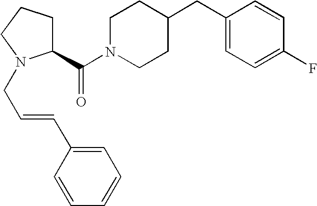 | 407.1 |
| 270 | 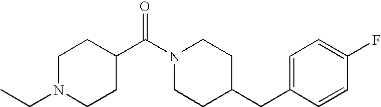 | 333.1 | 271 | 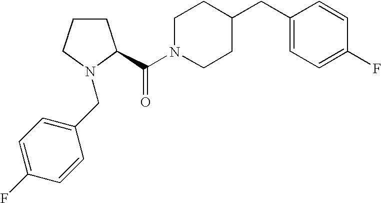 | 399.1 |
| 272 | 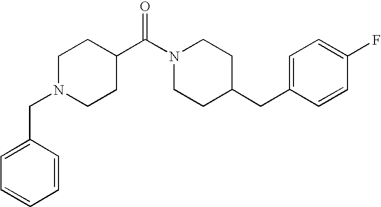 | 395.2 | 273 | 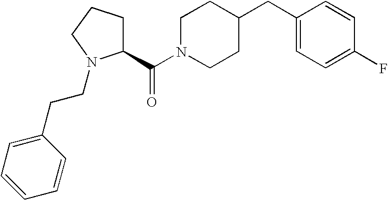 | 395.1 |
| 274 | 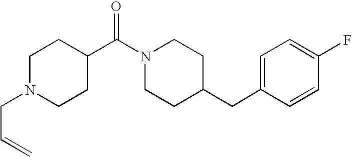 | 345.1 | 275 | 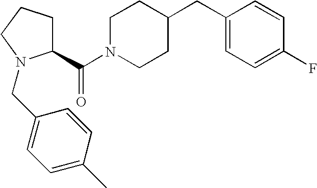 | 395.1 |
| 276 | 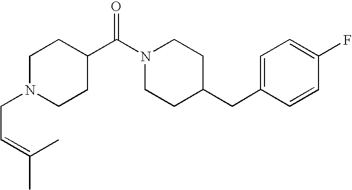 | 373.2 | 277 | 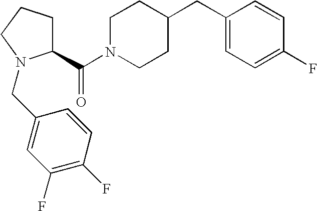 | 417.1 |
| 278 | 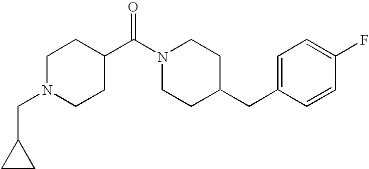 | 359.2 | 279 | 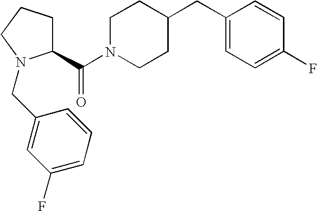 | 399.1 |

### EXAMPLE 66

Preparation of ((2S)-1-Ethylpiperidin-2-yl)-[4-(4-fluoro Benzylidene)piperidin-1-yl]methanone Hydrochloride (Compound 84)

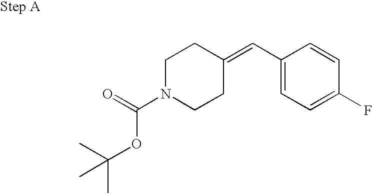

4-(4-Fluorobenzylidene)piperidine-1-carboxylic Acid Tert-Butyl Ester (Compound 82).

4-Fluorobenzyl triphenylphosphonium chloride (54.2 g, 133.2 mmol) was suspended in 400 ml of anhydrous THF. Sodium hydride (60% dispersion in mineral oil; 5.35 g, 133.2 mmol) was added to the suspension and stirred at room temperature for 3 hours. A solution of tert-butyl 4-oxo-1-piperidinecarboxylate (25 g, 125.5 mmol) in 150 ml of anhydrous THF was added dropwise over 1 hour. The reaction was heated to reflux for 8 hours and then cooled to room temperature, filtered, and the filtrate evaporated in vacuo to afford the crude product as a yellow viscous oil. The crude product was purified by flash chromatography (SiO2) eluted with a gradient of hexane to hexane-ethyl acetate (7:3). The pure fractions were combined and evaporated to afford 25.83 g (70% yield) of Compound 82 as a white crystalline solid.

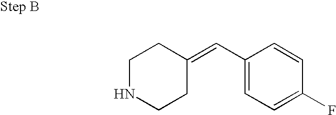

4-(4-Fluorobenzylidene)piperidine hydrochloride (Compound 83).

4-(4-Fluorobenzylidene)piperidine-1-carboxylic acid tert-butyl ester (Compound 82; 695 mg, 2.38 mmol) was dissolved in 25 ml of ethyl acetate and anhydrous HCl gas was bubbled into the solution at room temperature until warm. The reaction was stirred for 1 hour, then evaporated in vacuo to afford 521 mg (96% yield) of the desired product as a white crystalline solid.

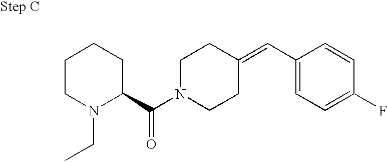

((2S)-1-Ethylpiperidin-2-yl)-[4-(4-fluorobenzylidene)piperidin-1-yl]-methanone hydrochloride (Compound 84).

Compound 84 was prepared from (2S)-1-ethylpiperidin-2-yl carboxylic acid and 4-(4-Fluorobenzylidene)piperidine hydrochloride (Compound 83) as described in Example 1 to yield 234 mg (70%) as the hydrochloride salt.

¹H NMR (CD₃OD, 500 MHz) δ 7.23 (m, 2H), 7.05 (m, 2H), 6.48 (s, 1H), 4.56 (m, 1H), 3.84 (m, 0.5H), 3.72 (m, 2H), 3.65 (m, 2H), 3.55 (m, 0.5H), 3.23 (m, 1H), 3.04 (m, 2H), 2.61 (m, 1H), 2.53 (m, 2H), 2.44 (m, 1H), 2.18 (m, 1H), 1.96 (m, 2H), 1.88-1.68 (m, 3H), 1.38 (t, 3H). MS m/z 331.04 (M+1)

### EXAMPLE 67

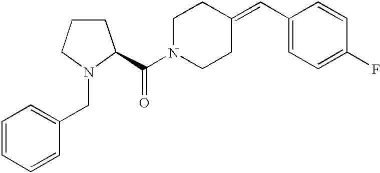

((2S)-1-Benzylpyrrolidin-2-yl)-[4-(4-fluorobenzylidene) piperidin-1-yl]methanone Hydrochloride (Compound 85).

Compound 85 was prepared from (2S)-1-benzyl-pyrrolidin-2-yl carboxylic acid and 4-(4-Fluorobenzylidene)piperidine hydrochloride (Compound 83) as described in Example 1 to yield 310 mg (79%) as the hydrochloride salt.

¹H NMR (CD₃OD, 500 MHz) δ 7.57 (m, 2H), 7.48 (m, 3H), 7.22 (m, 2H), 7.08 (m, 2H),-6.46 (m, 1H), 4.79 (m, 1H), 4.50 (d, 1H), 4.32 (d, 1H), 3.71 (m, 1.5H), 3.62 (m, 0.5H), 3.48-3.21 (m, 3.5H), 2.65 (m, 1H), 2.52 (m, 1H), 2.42-2.22 (m, 3H), 2.12 (m, 1H), 2.05 (m, 1H), 1.95 (m, 1H). MS m/z 379.12 (M+1)

### EXAMPLE 70

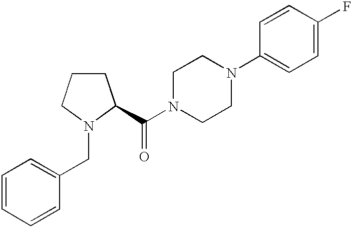

((2S)-1-Benzylpyrrolidin-2-yl)-[4-(4-fluorophenyl) piperazin-1-yl]methanone Hydrochloride (Compound 86).

Compound 86 was prepared from (2S)-1-benzylpyrrolidin-2-yl carboxylic acid and 4-(4-fluoro-phenyl)piperazine as described in Example 1 to yield 620 mg (72%) as the dihydrochloride salt.

¹H NMR (CDCl₃, 500 MHz) δ 7.34 (m, 5H), 7.02 (m, 2H), 6.84 (m, 2H), 4.02 (m, 1H), 3.96-3.68 (m, 4H), 3.55 (m, 2H), 3.26-2.95 (m, 5H), 2.38 (m, 1H), 2.21 (m, 1H), 1.92 (m, 3H). MS m/z 368.3 (M+1)

### EXAMPLE 71

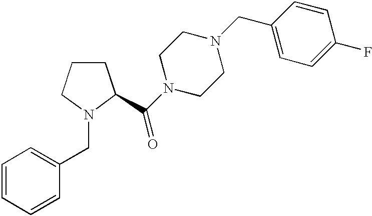

((2S)-1-Benzyl-pyrrolidin-2-yl)-[4-(4-fluoro-benzyl)-piperazin-1-yl]methanone (Compound 87).

Compound 87 was prepared from (2S)-1-benzylpyrrolidin-2-yl carboxylic acid and 4-(4-fluorobenzyl)piperazine as described in Example 1 to yield 210 mg (36% yield) as the dihydrochloride salt.

¹H NMR (CDCl₃, 500 MHz) 7.25 (m, 7H), 6.95 (m, 2H), 3.90 (d, 1H), 3.65-3.49 (m, 4H), 3.41 (s, 2H), 3.31. (m, 1H), 2.97 (m, 1H), 2.25 (m, 6H), 2.13 (m, 1H), 1.81 (m, 2H), 1.72 (m, 1H). MS m/z 382.16 (M+1).

### EXAMPLE 72

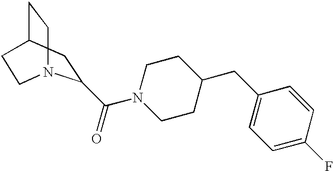

(1-Aza-bicyclo[2.2.2]oct-2-yl)-[4-(4-fluoro-benzyl)-piperidin-1-yl]methanone hydrochloride (Compound 88).

Compound 88 was prepared from 1-azabicyclo[2.2.2]octane-2-carboxylic acid and 4-(4-fluorobenzyl)piperidine as described in Example 1 to yield 30 mg (19%) as the hydrochloride salt.

¹H NMR (CDCl₃, 500 MHz) 7.09 (m, 2H), 6.95 (m, 2H), 4.61 (d, 1H), 4.01-3.88 (m, 2H), 3.49 (s, 1H), 3.41 (s, 1H), 3.21-3.03 (m, 2H), 2.92 (m, 1H), 2.55 (m, 3H), 2.25 (m, 1H), 2.05 (d, 1H), 1.80-1.55 (m, 7H), 1.39 (m, 1H), 1.15 (m, 2H). MS m/z 331.08 (M+1).

### EXAMPLE 73

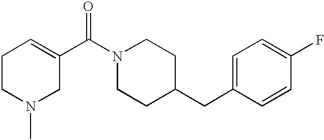

[4-(4-Fluorobenzyl)piperidin-1-yl]—(1-methyl-1,2,5,6-tetrahydropyridin-3-yl) Methanone Hydrochloride (Compound 89).

Compound 89 was prepared from arecaidine hydrochloride and 4-(4-fluorobenzyl)piperidine as described in Example 33 to yield 1.26 g (91%) as the hydrochloride salt.

¹H NMR (CD₃OD, 500 MHz) δ 7.08 (m, 2H), 6.88 (m, 2H), 6.04 (s, 1H), 4.28 (m, 1H), 4.02 (m, 1H), 3.93 (d, 1H), 3.67 (d, 1H), 3.48 (m, 1H), 3.12 (m, 1H), 2.95 (m, 1H), 2.86 (s, 3H), 2.56 (m, 2H), 2.47 (m, 3H), 1.73 (m, 1H), 1.62 (m, 2H), 1.06 (m, 2H). MS m/z 317.2 (M+1).

### EXAMPLE 74

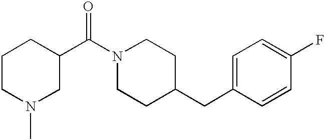

[4-(4-Fluorobenzyl)piperidin-1-yl]-(1-methylpiperidin-3-yl) Methanone Hydrochloride (Compound 90).

Compound 89 (200 mg) was dissolved in 10 ml of ethanol. To the solution was added 50 mg of 10% palladium on carbon and the flask charged with an atmosphere of hydrogen (1 atm.). After 3 hours, the reaction was filtered through Celite and evaporated to afford compound 90 as a clear viscous oil which was converted to the hydrochloride salt (132 mg).

¹H NMR (CD3OD, 500 MHz) δ 7.07 (m, 2H), 6.88 (m, 2H), 4.43 (d, 0.5H), 4.38 (d, 0.5H), 3.88 (d, 0.5H), 3.76 (d, 0.5H), 3.50-3.22 (m, 3H), 3.18 (s, 0.5H), 3.10 (m, 0.5H), 2.98 (m, 2H), 2.85 (m, 1H), 2.78 (m, 1H), 2.53 (m, 1H), 2.48 (m, 2H), 1.94-1.34 (m, 7H), 1.3-0.92 (m, 3H). MS m/z 319.3 (M+1).

### EXAMPLE 75

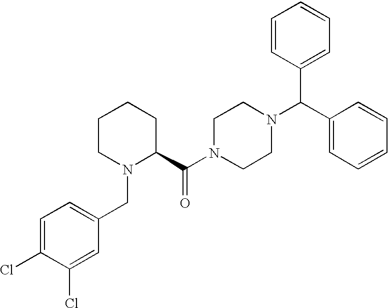

(4-Benzhydryl-piperazin-1-yl)-[(2S)1-(3,4-dichloro-benzyl)-piperidin-2-yl]methanone Dihydrochloride (Compound 91).

Compound 91 was prepared from 1-[4-(1,1-diphenylmethyl)piperazin-1-yl]-(2S)-piperidin-2-yl methanone dihydrochloride and 3,4-dichlorobenzyl chloride as described for Compound 21 in Example 9 to afford 56 mg (56%) as the dihydrochloride salt.

¹H NMR (CDCl₃, 500 MHz) 7.48-7.25 (m, 10H), 7.21 (d, 2H), 7.15 (m, 1H), 4.21 (s, 1H), 3.81 (d, 2H), 3.65 (s, 2H), 3.24 (m, 2H), 2.91 (s, 1H), 2.38 (s, 4H), 1.98 (s, 1H), 1.75 (s, 3H), 1.51 (s, 2H), 1.29 (s, 2H). MS m/z 523.01 (M+1).

### EXAMPLE 76

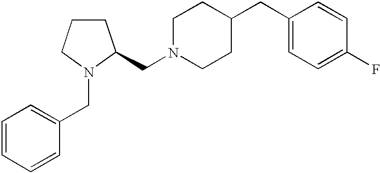

1-((2S)-1-Benzylpyrrolidin-2-ylmethyl)-4-(4-fluorobenzyl)piperidine Dihydrochloride (Compound 103).

Compound 103 was prepared by the reduction of Compound 49 as described in Example 60 to yield 241 mg (89%) of the title compound as the dihydrochloride salt.

¹H NMR (CDCl₃, 500 MHz) δ 7.38-7.30 (m, 5H), 7.09 (m, 2H), 6.98 (m, 2H), 4.3 (d, 1H), 3.33 (m, 1H), 2.97 (br s, 3H), 2.66 (m, 2H), 2.52 (d, 2H), 2.37 (br s, 1H), 2.18 (br s, 1H), 1.98 (m, 3H), 1.85-1.55 (m, 5H), 1.51 (m, 1H), 1.32 (m, 2H). MS m/z 367.4 (M+1).

### EXAMPLE 77

General Methods

The ventral mesencephalic region was dissected out of embryonic day 15 Sprague-Dawley rat embryos (Harlan), dissociated into single cell suspension by a combination of trypsinization and trituration (Costantini et al., Neurobiol Dis. 1998; 5:97-106). Dissociated VM cells were plated into poly-L-ornithine-coated 60-mm dishes at a density of 5.6×10⁶ cells/dish in 6 mL of DMEM supplemented with 18% heat-inactivated horse serum, 0.24% glucose, 2 mM glutamine and 50 u/ml pernicillin/streptomycin and incubated in a 5% CO₂ incubator. After one day in culture, the medium was replaced with 6 mL of a defined medium (DMEM supplemented with 1×N2 cocktail (Gibco-BRL), 0.12% glucose, 2 mM glutamine, and 50 units/ml penicillin/streptomycin) containing 0.05% DMSO (vehicle control), 312 nM VRT-104136 or the same concentration of VRT-104953. Cells were harvested at 0 (before compound addition), 1, 3, 4, and 5 days after the addition of compounds of the present invention by scrapping them into Hepes-buffered saline (HBS) with a rubber policeman. Cells harvested after 5 days of compound treatment were also treated with 400 uM NMDA for 20 hour prior to harvest. Scrapped cells were pelleted by gentle centrifugation and the cell pellets were frozen in liquid nitrogen and stored at −80 C till use. A separate VM preparation was used for each time point and 6-8 dishes of cells were used for each condition.

Total RNA was isolated from VM cells using the RNeasy total RNA preparation kit (Qiagen) according to manufacture's recommended procedures.

| Time | | | | | |
|---|---|---|---|---|---|
| after | | | | | |
| compound | | | | | total |
| addition | | OD260 | | vol | RNA |
| (day) | treatment | (1/50) | ug/mL | (uL) | (ug) |
| 0 | none | 0.2255 | 451 | 48 | 21.6 |
| 1 | DMSO | 0.1194 | 238.8 | 48 | 11.5 |
| | 312- | 0.1592 | 318.4 | 48 | 15.3 |
| | 104136 | | | | |
| | 312- | 0.1248 | 249.6 | 48 | 12.0 |
| | 104953 | | | | |
| 3 | DMSO | 0.2209 | 441.8 | 48 | 21.2 |
| | 312- | 0.2466 | 493.2 | 48 | 23.7 |
| | 104136 | | | | |
| | 312- | 0.2901 | 580.2 | 48 | 27.8 |
| | 104953 | | | | |
| 4 | DMSO | 0.4012 | 802.4 | 48 | 38.5 |
| | 312- | 0.353 | 706 | 48 | 33.9 |
| | 104136 | | | | |
| | 312- | 0.3919 | 783.8 | 48 | 37.6 |
| | 104953 | | | | |
| 5 | DMSO | 0.2892 | 578.4 | 48 | 27.8 |
| | 312- | 0.3488 | 697.6 | 48 | 33.5 |
| | 104136 | | | | |
| | 312- | 0.3738 | 747.6 | 48 | 35.9 |
| | 104953 | | | | |

### EXAMPLE 78

Compound Treatment and RNA Preparation from VM Culture

The ventral mesencephalic region was dissected out of embryonic day 15 Sprague-Dawley rat embryos (Harlan), dissociated into single cell suspension by a combination of trypsinization and trituration (Costantini et al., Neurobiol Dis. 1998; 5:97-106). Dissociated VM cells were plated into poly-L-ornithine-coated 60-mm dishes at a density of 5.6×10⁶ cells/dish in 6 mL of DMEM supplemented with 18% heat-inactivated horse serum, 0.24% glucose, 2 mM glutamine and 50 u/ml pernicillin/streptomycin and incubated in a 5% CO₂ incubator. After one day in culture, the medium was replaced with 6 mL of a defined medium (DMEM supplemented with 1×N2 cocktail (Gibco-BRL), 0.12% glucose, 2 mM glutamine, and 50 units/ml penicillin/streptomycin) containing 0.05% DMSO (vehicle control), 312 nM VRT-104136 or the same concentration of VRT-104953. Cells were harvested at 0 (before compound addition), 1, 3, 4, and 5 days after the addition of neurophilin compounds by scrapping them into Hepes-buffered saline (HBS) with a rubber policeman. Cells harvested after 5 days of compound treatment were also treated with 400 uM NMDA for 20 hour prior to harvest. Scrapped cells were pelleted by gentle centrifugation and the cell pellets were frozen in liquid nitrogen and stored at −80 C till use. A separate VM preparation was used for each time point and 6-8 dishes of cells were used for each condition.

Total RNA was isolated from VM cells using the RNeasy total RNA preparation kit (Qiagen) according to manufacture's recommended procedures.

| Time after | | | | | |
|---|---|---|---|---|---|
| compound | | OD260 | | vol | total |
| addition (day) | treatment | (1/50) | ug/mL | (uL) | RNA (ug) |
| 0 | none | 0.2255 | 451 | 48 | 21.6 |
| 1 | DMSO | 0.1194 | 238.8 | 48 | 11.5 |
| | 312-104136 | 0.1592 | 318.4 | 48 | 15.3 |
| | 312-104953 | 0.1248 | 249.6 | 48 | 12.0 |
| 3 | DMSO | 0.2209 | 441.8 | 48 | 21.2 |
| | 312-104136 | 0.2466 | 493.2 | 48 | 23.7 |
| | 31 2-1 04953 | 0.2901 | 580.2 | 48 | 27.8 |
| 4 | DMSO | 0.4012 | 802.4 | 48 | 38.5 |
| | 312-104136 | 0.353 | 706 | 48 | 33.9 |
| | 312-104953 | 0.3919 | 783.8 | 48 | 37.6 |
| 5 | DMSO | 0.2892 | 578.4 | 48 | 27.8 |
| | 312-104136 | 0.3488 | 697.6 | 48 | 33.5 |
| | 312-104953 | 0.3738 | 747.6 | 48 | 35.9 |

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

While we have described a number of embodiments of this invention, it is apparent that our basic examples may be altered to provide other embodiments which utilize the compounds and methods of this invention. Therefore, it will be appreciated that the scope of this invention is to be defined by the appended claims rather than by the specific embodiments which have been represented by way of example.

We claim:

1. A method of modulating cholesterol biosynthesis in a mammal by administering to said mammal a composition comprising:
(a) a compound of formula (I): 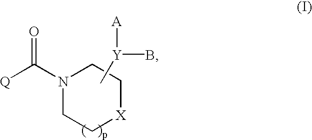
wherein:
each Q is a monocyclic, bicyclic or tricyclic ring system wherein in said ring system:
a. each ring is independently partially unsaturated or fully saturated;
b. each ring comprises 3 to 7 ring atoms independently selected from C, N, O or S;
c. no more than 4 ring atoms in Q are selected from N, O or S;
d. any S is optionally replaced with S(O) or S(O)₂;
e. at least one ring comprises a N ring atom that is substituted with R¹; and
f. one to five hydrogen atoms in Q are optionally and independently replaced with halo, —OH, ═O, ═N—OR¹, (C₁-C₆)-straight or branched alkyl, Ar-substituted-(C₁-C₆)-straight or branched alkyl, (C₂-C₆)-straight or branched alkenyl or alkynyl, Ar-substituted-(C₂-C₆)-straight or branched alkenyl or alkynyl, O—(C₁-C₆)-straight or branched alkyl, O-[(C₁-C₆)-straight or branched alkyl]-Ar, O—(C₂-C₆)-straight or branched alkenyl or alkynyl, O-[(C₂-C₆)-straight or branched alkenyl or alkynyl]-Ar, or O—Ar; wherein
each R¹ is independently selected from (C₁-C₆)-straight or branched alkyl, Ar-substituted-(C₁-C₆)-straight or branched alkyl, cycloalkyl-substituted-(C₁-C₆)-straight or branched alkyl, (C₂-C₆)-straight or branched alkenyl or alkynyl, or Ar-substituted-(C₂-C₆)-straight or branched alkenyl or alkynyl; wherein
one to two CH₂ groups of said alkyl, alkenyl, or alkynyl chains in R¹ are optionally and independently replaced with O, S, S(O), S(O)₂, C(O) or N(R²), wherein when R¹ is bound to nitrogen, the CH₂ group of R¹ bound directly to said nitrogen cannot be replaced with C(O);
Ar is selected from phenyl, 1-naphthyl, 2-naphthyl, indenyl, azulenyl, 2-furyl, 3-furyl, 2-thienyl, 3-thienyl, 2-pyridyl, 3-pyridyl, 4-pyridyl, pyrrolyl, oxazolyl, thiazolyl, imidazolyl, pyraxolyl, pyrazolinyl pyraolidinyl, isoxazolyl, isothiazolyl, 1,2,3-oxadiazolyl, 1,2,3-triazolyl, 1,3,4-thiadiazolyl, 1,2,4-triazolyl, 1,2,4-oxadiazolyl, 1,2,4-thiadiazolyl, 1,2,3-thiadiazolyl, benoxazolyl, pyridazinyl, 2-pyrimidinyl, 4-pyrimidinyl, 5-pyrimidinyl, pyrazinyl, 1,3,5-triazinyl, 1,3,5-trithianyl, indolizinyl, indolyl, isoindolyl, 3H-indolyl, indolinyl, benzo[b]furanyl, benzo[b]thiophenyl, 1H-indazolyl, benzimidazolyl, benzthiazolyl, purinyl, 4H-quinolizinyl, quinolinyl, 1,2,3,4-tetrahydroisoquinolinyl, isoquinolinyl, 1,2,3,4-tetrahydroquinolinyl, cinnolinyl, phthalazinyl, quinazolinyl, quinoxalinyl, 1,8-naphthyridinyl, or any other chemically feasible monocyclic or bicyclic ring system, wherein each ring consists of 5 to 7 ring atoms and wherein each ring comprises 0 to 3 heteroatoms independently selected from N, O, or S, wherein
each Ar is optionally and independently substituted with one to three substituents selected from halo, hydroxy, nitro, —SO₃H, ═O, trifluoromethyl, trifluoromethoxy, (C₁-C₆)-straight or branched alkyl, (C₁-C₆)-straight or branched alkenyl, O-[(C₁-C₆)-straight or branched alkyl], O-[(C₁-C₆)-straight or branched alkenyl], O-benzyl, O-phenyl, 1,2-methylenedioxy, —N(R³) (R⁴), carboxyl, N-(C₁-C₆-straight or branched alkyl or C₂-C₆-straight or branched alkenyl) carboxamides, N,N-di-(C₁-C₆-straight or branched alkyl or C₂-C₆-straight or branched alkenyl) carboxamides, N-(C₁-C₆-straight or branched alkyl or C₂-C₆-straight or branched alkenyl) sulfonamides, or N,N-di-(C₁-C₆-straight or branched alkyl or C₂-C₆-straight or branched alkenyl) sulfonamides;
each of R³ and R⁴ are independently selected from (C₁-C₆)-straight or branched alkyl, (C₂-C₆)-straight or branched alkenyl or alkynyl, hydrogen, phenyl or benzyl; or wherein R³ and R⁴ are taken together with the nitrogen atom to which they are bound to form a 5-7 membered heterocyclic ring;
R² is selected from hydrogen, (C₁-C₆)-straight or branched alkyl, or (C₂-C₆)-straight or branched alkenyl or alkynyl;
X is selected from C, N(R²), N, O, S, S(O), or S(O)₂
Y is selected from a bond, —O—, (C₁-C₆)-straight or branched) alkyl, or (C₂-C₆)-straight or branched) alkenyl or alkynyl; wherein Y is bonded to the depicted ring via a single bond or a double bond; and wherein one to two of the CH₂ groups of said alkyl, alkenyl, or alkynyl is optionally and independently replaced with O, S, S(O), S(O)₂, C(O) or N(R);
p is 0, 1 or 2;
each of A and B is independently selected from hydrogen or Ar; and
wherein two carbon ring atoms in the depicted ring structure may be linked to one another via a C₁-C₄ straight alkyl or a C₂-C₄ straight alkenyl to create a bicyclic moiety; and
(b) a pharmaceutically acceptable carrier.

2. A method for treating a disease mediated by cholesterol biosynthesis comprising the step of administering to a patient a composition according to claim 1.

3. The method according to claim 2, wherein said disease is Creutzfeld-Jakob disease, including the sporadic, inherited and the infectious forms, bovine spongiform encephalopathy, scrapie, Niemann-Pick Type C disease, Smith-Lemli-Opitz syndrome or Tangier disease.

4. The method according to claim 3, wherein said disease is Creutzfeldt-Jakob disease, including the sporadic, inherited and the infectious forms, bovine spongiform encephalopathy or scrapie.

5. The method according to claim 3, wherein said disease is Creutzfeldt-Jakob disease, including the sporadic, inherited and the infectious forms.

6. The method according to claim 2, wherein said disease is a veterinary disease selected from BSE, scrapie and transmissible mink encephalopathy, including the sporadic, inherited and the infectious forms.

7. The method according to any one of claims 1-6, wherein said compound is selected from any one of Table 1, Table 2 or Table 3.
